(19) United States
(12) Patent Application Publication
(10) Pub. No.: US 2020/0381078 A1
(43) Pub. Date: Dec. 3, 2020

(54) STRUCTURE SEARCH APPARATUS, METHOD, AND RECORDING MEDIUM

(71) Applicant: FUJITSU LIMITED, Kawasaki-shi (JP)

(72) Inventor: Mari Ohfuchi, Hadano (JP)

(73) Assignee: FUJITSU LIMITED, Kawasaki-shi (JP)

(21) Appl. No.: 16/883,314

(22) Filed: May 26, 2020

(30) Foreign Application Priority Data
May 30, 2019 (JP) 2019-101216

(51) Int. Cl. G16B 15/00; G16B 40/00; G06F 17/11

(52) U.S. Cl. CPC G16B 15/00; G06F 17/11; G16B 40/00

(57) ABSTRACT

A structure formed with a plurality of molecules that have interactions is searched by a computer. The computer performs a process including: preparing position bits of a number calculated based on the number of constituent units included in the plurality of molecules and the number of molecules included in the plurality of molecules for each constituent unit in each molecule included in the plurality of molecules, and searching the structure formed with the plurality of molecules that have the interactions based on a cost function that includes (A-1) a negative interaction, (A-2) an original interaction, (B-1) a constraint that each of the constituent units in the one molecule exists in one of the position bits, and (B-2) a constraint that one or no constituent unit exists in any one of the plurality of molecules in the position bits.

### CROSS-REFERENCE TO RELATED APPLICATION

This application is based upon and claims the benefit of priority of the prior Japanese Patent Application No. 2019-101216, filed on May 30, 2019, the entire contents of which are incorporated herein by reference.

### FIELD

The embodiment discussed herein is related to a structure search apparatus, a structure search method, and a recording medium.

### BACKGROUND

In recent years, in a scene such as a drug discovery, it may be unavoidable to obtain a stable structure of a molecule having a large size by using a calculator (a computer). However, for example, in a case of a molecule formed with a large number of atoms, it may be difficult to search for a stable structure within a practical time in a calculation in consideration of a state where all atoms are exposed.

As a technique for coarse graining of a molecular structure, for example, there has been studied a technique in which a molecular structure is subjected to coarse graining into a linear (one series) simple cubic lattice structure based on one-dimensional sequence information of an amino acid residue in a protein and is treated as a lattice protein. There has been reported a technique for searching for a stable structure at high speed by using a technique of quantum annealing in a lattice protein (see, for example, Alejandro Perdomo-Ortiz et. al., “Finding low energy conformations of lattice protein models by quantum annealing”, Scientific Reports, volume 2, Article number: 571, 2012, and Ryan Babbush et al., “Construction of Energy Functions for Lattice Heteropolymer Models: Efficient Encodings for Constraint Satisfaction Programming and Quantum Annealing”, Advances in Chemical Physics, 155, 201-243.

In these techniques for searching for the stable structure of the lattice protein by using an annealing machine, a starting point of the lattice is positioned, and an advancing direction of the amino acid residue or an adjacent position to the amino acid residue is expressed by a bit (0 or 1). Therefore, these techniques of related art are techniques capable of searching for only a structure formed with one molecule coupled in a linear chain state, and may not be used to search a structure formed with a plurality of molecules such as a polymer aggregate.

In one aspect, it is an object of the present disclosure to provide a structure search apparatus, a structure search method, and a computer-readable recording medium in which a structure search program is stored that are capable of searching a structure formed with a plurality of molecules.

### SUMMARY

According to an aspect of the embodiments, a structure search method includes; preparing position bits of a number calculated based on the number of constituent units included in the plurality of molecules and the number of molecules included in the plurality of molecules for each constituent unit in each molecule included in the plurality of molecules, and searching the structure formed with the plurality of molecules that have the interactions based on a cost function that includes (A-1) a negative interaction, (A-2) an original interaction, (B-1) a constraint that each of the constituent units in the one molecule exists in one of the position bits, and (B-2) a constraint that one or no constituent unit exists in any one of the plurality of molecules in the position bits.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1A is a schematic diagram illustrating an example in which a protein is searched for a stable structure by using a coarse graining procedure (Part 1);

FIG. 1B is a schematic diagram illustrating an example in which a protein is searched for a stable structure by using a coarse graining procedure (Part 2);

FIG. 1C is a schematic diagram illustrating an example in which a protein is searched for a stable structure by using a coarse graining procedure (Part 3);

FIG. 2A is a schematic diagram for explaining an example of a turn encoding method (Part 1);

FIG. 2B is a schematic diagram for explaining an example of the turn encoding method (Part 2);

FIG. 2C is a schematic diagram for explaining an example of the turn encoding method (Part 3);

FIG. 2D is a schematic diagram for explaining an example of the turn encoding method (Part 4);

FIG. 3A is a schematic diagram for explaining an example of a diamond encoding method (Part 1);

FIG. 3B is a schematic diagram for explaining an example of the diamond encoding method (Part 2);

FIG. 3C is a schematic diagram for explaining an example of the diamond encoding method (Part 3);

FIG. 3D is a schematic diagram for explaining an example of the diamond encoding method (Part 4);

FIG. 3E is a schematic diagram for explaining an example of the diamond encoding method (Part 5);

FIG. 4 is a schematic diagram illustrating an example of a structure of a block copolymer;

FIG. 5 is a schematic diagram for explaining an example of a periodic boundary condition in an example of the technique disclosed herein;

FIG. 6A is a schematic diagram illustrating an example of a relationship between a constituent unit and a position bit;

FIG. 6B is a schematic diagram illustrating another example of the relationship between the constituent unit and the position bit;

FIG. 7A is a schematic diagram illustrating an example of interactions in searching for a structure in which two molecules AB composed of constituent units A and B are present (Part 1);

FIG. 7B is a schematic diagram illustrating an example of interactions in searching for a structure in which two molecules AB composed of constituent units A and B are present (Part 2);

FIG. 8 is a schematic diagram illustrating an example of a position bit prepared for each constituent unit;

FIG. 9 is a schematic diagram illustrating an example of a structure in which constituent units in one molecule are not separated from each other and are arranged one by one in each position bit without overlapping with each other;

FIG. 10 is a diagram illustrating a hardware configuration example of a structure search apparatus disclosed herein;

FIG. 11 is a diagram illustrating another hardware configuration example of the structure search apparatus disclosed herein;

FIG. 12 is a diagram illustrating another hardware configuration example of the structure search apparatus disclosed herein;

FIG. 13 is a diagram illustrating a functional configuration example of the structure search apparatus disclosed herein;

FIG. 14 is a diagram illustrating an example of a weight file;

FIG. 15 is an example of a flowchart in searching a structure formed with a plurality of molecules by using an example of the technique disclosed herein;

FIG. 16 is an example of a flowchart when a structure at a plurality of desired temperatures (one temperature) is searched and an energy of the structure is calculated, by using an example of the technique disclosed herein;

FIG. 17 is a diagram illustrating an example of a functional configuration of an optimization apparatus (control unit) to be used in an annealing method;

FIG. 18 is a block diagram illustrating an example of a transition control unit at a circuit level;

FIG. 19 is a diagram illustrating an example of an operation procedure of the transition control unit;

FIG. 20 is a schematic diagram illustrating a search result of a structure in Example 1-1;

FIG. 21 is a schematic diagram illustrating a search result of a structure in Example 1-2;

FIG. 22 is a diagram illustrating an average value of energies for each temperature in Examples 2-1 and 2-2; and

FIG. 23 is a diagram illustrating an example of the structure search of a molecule in each of Examples of the technique disclosed herein and the technique of related art.

### DESCRIPTION OF EMBODIMENTS

(Structure Search Apparatus)

The structure search apparatus disclosed herein is an apparatus for searching a structure formed with a plurality of molecules having interactions. The structure search apparatus disclosed herein includes a structure search unit, and further includes other units (means) as appropriate.

Before describing details of the technique disclosed herein, a method for determining a folding structure of a protein is described by using a technique using a lattice protein as the technique of the related art.

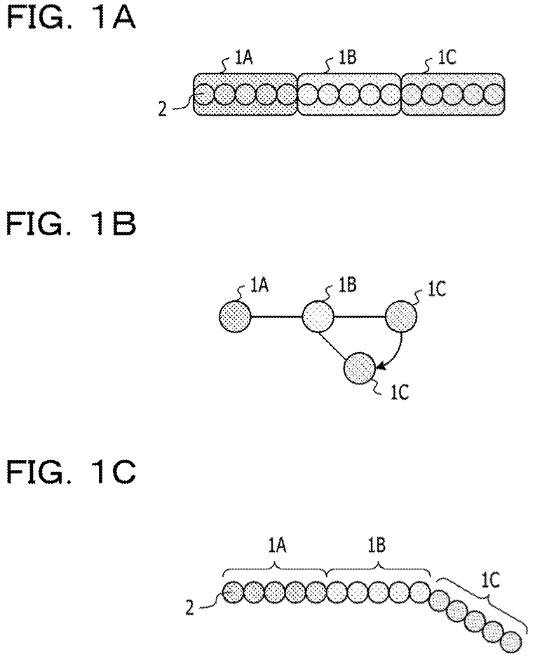

First, a turn encoding method which is one of techniques using a lattice protein will be described. When a structure search of a protein (or peptide) is performed by using the lattice protein, coarse graining of the protein is firstly performed. As illustrated in FIG. 1A, the coarse graining of the protein is performed, for example, by creating a coarse-grained model by performing the coarse graining on atoms 2 constituting the protein into coarse-grained particles 1A, 1B, and 1C each of which is a unit for each amino acid residue.

Next, the created coarse-grained model is used to search for a stable binding structure. FIG. 1B illustrates an example of a case where the binding structure in which the coarse-grained particle 1C is located at an end point of an arrow is stable. The stable binding structure is searched by a turn encoding method or a diamond encoding method described later. As illustrated in FIG. 1C, the coarse-grained model is returned to a model for all atoms based on the searched stable structure.

In the turn encoding method, in many cases, when particles (coarse-grained model) obtained by performing the coarse graining on a chain amino acid forming a protein are applied to lattice points of a lattice, a position serving as a starting point in the lattice is determined to express an advancing direction of an amino acid residue by a bit. In the following description, for simplification of explanation, the turn encoding method applied to a two-dimensional case will be described as an example.

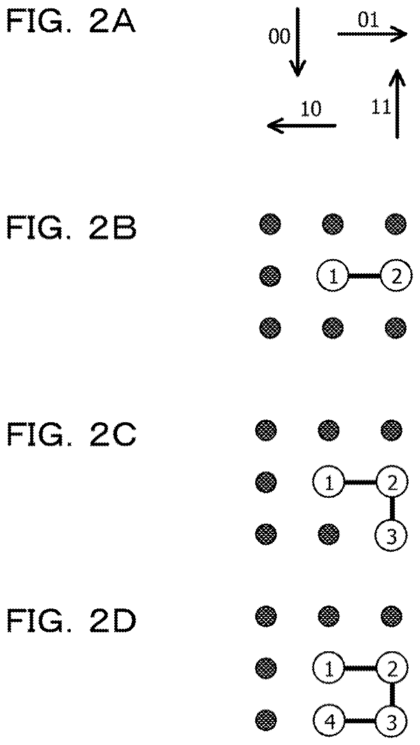

A case where an advancing direction of an amino acid residue in a two-dimensional lattice is defined by two bits (2 bits), for example, as illustrated in FIG. 2A, will be considered. In the example illustrated in FIG. 2A, for example, it is represented that when the bits are [00], the amino acid residue advances (is bound) downward, and when the bits are [01], the amino acid residue advances to a right direction. Similarly, in the example illustrated in FIG. 2A, for example, it is represented that when the bits are [10], the amino acid residue advances to a left direction, and when the bits are [11], the amino add residue advances upward.

In the turn encoding method, as illustrated in FIG. 2B, in a case where an amino acid residue having the number 1 is arranged in a center of the lattice, when an amino acid residue having the number 2 is arranged at a lattice point on a right side of the amino acid residue having the number 1, the bits representing the advancing direction of the amino acid residue are [01].

Next, as illustrated in FIG. 2C, when an amino add residue having the number 3 is arranged at a lattice point under the amino acid residue having the number 2, the bits representing the advancing directions of the amino add residues are [0100].

Subsequently, as illustrated in FIG. 2D, when an amino add residue having the number 4 is arranged at a lattice point on a left side of the amino acid residue having the number 3, the bits representing the advancing directions of the amino acid residues are [010010].

In this way, in the turn encoding method, it is possible to express the structure of the coarse-grained protein by positioning the starting point in the lattice and expressing the advancing directions of the amino acid residues by the bits.

Next, as another example of the technique using the lattice protein, the diamond encoding method will be described. For example, the diamond encoding method is a method of fitting a particle (coarse-grained model) subjected to coarse graining on a chain amino acid forming a protein to a lattice point of a diamond lattice, and it is possible to express a three-dimensional structure of a protein. In the following description, for simplification of explanation, the diamond encoding method applied to a two-dimensional case will be described as an example.

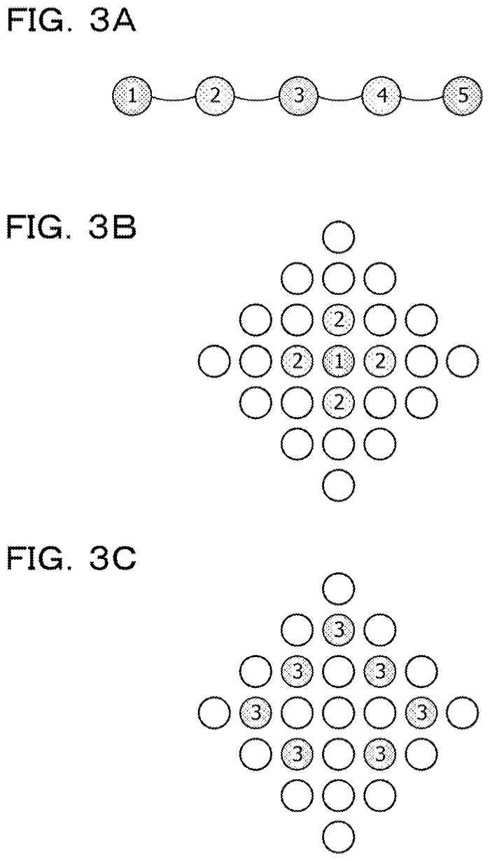

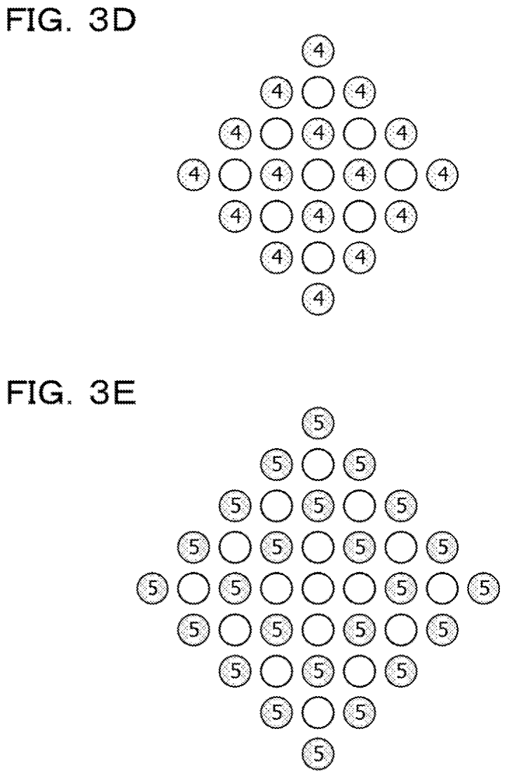

FIG. 3A illustrates an example of a structure in which a linear pentapeptide having five amino add residues bound to each other has a linear structure. In FIG. 3A to FIG. 3E, each of numbers in circles represents a number of an amino acid residue in the linear pentapeptide.

In the diamond encoding method, first, when an amino acid residue having the number 1 is arranged at a center of a diamond lattice, as illustrated in FIG. 31, a place where an amino acid residue having the number 2 may be arranged is limited to places adjacent to the center (places where the number 2 is given).

Subsequently, a place where an amino acid residue having the number 3 to be bound to the amino acid residue having the number 2 may be arranged is, in FIG. 3C, limited to places adjacent to the places where the number 2 is given in FIG. 3B (places where the number 3 is given).

A place where an amino acid residue having the number 4 to be bound to the amino acid residue having the number 3 may be arranged is, in FIG. 3D, limited to places adjacent to the places where the number 3 is given in FIG. 3C (places where the number 4 is given).

A place where an amino acid residue having the number 5 to be bound to the amino acid residue having the number 4 may be arranged is, in FIG. 3E, limited to places adjacent to the places where the number 4 is given in FIG. 3D (places where the number 5 is given).

By linking the specified places where the amino acid residues may be arranged in the order of the amino acid residue numbers, the structure of the protein subjected to coarse graining may be expressed. For example, in the diamond encoding method, it is possible to express the structure of the protein subjected to coarse graining by positioning a starting point of the lattice and expressing an adjacent position to the amino acid residue by a bit.

However, the technique of the related art using these lattice proteins is a technique aimed at searching the structure of one protein, and it is not possible to search a structure formed with a plurality of molecules. For example, the above-described technique of the related art is a technique capable of searching only the structure formed with one molecule coupled in a linear chain state, and may not be used to search a structure formed with a plurality of molecules such as a polymer aggregate.

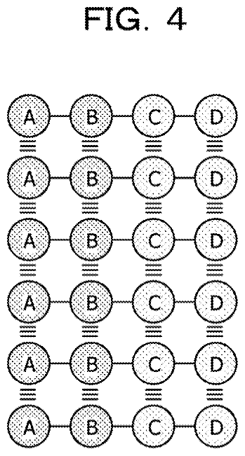

For example, in a block copolymer illustrated in FIG. 4, a plurality of molecules formed by binding particles A to D are present, and the molecules are separated into a layer formed with particles A and B and a layer formed with particles C and D due to an interaction of particles between different molecules. The structure of the plurality of molecules having such interactions may not be expressed by the technique of the related art in which the starting point of the lattice is positioned, and the advancing direction of the amino acid residue or the adjacent position to the amino add residue is expressed by a bit.

When a structure of a protein is searched, it may also be unavoidable to search a structure formed with a plurality of molecules. For example, many proteins have a structure in which a plurality of linear polypeptides (subunits) having a tertiary structure folded into a three-dimensional structure are bound to form a quaternary structure. For an expression of a function of a protein, a quaternary structure of the protein may be important, and in a case of a drug discovery or the like, there may be a case where accurately searching the quaternary structure of the protein is unavoidable.

However, since the quaternary structure of the protein is a structure formed with a plurality of polypeptides, the structure may not be searched in the above-described technique of the related art.

Therefore, the inventors of the present disclosure have extensively studied a device capable of searching a structure formed with a plurality of molecules, and have conceived the technique disclosed herein. For example, the inventors have prepared position bits of a number calculated based on the number of constituent units included in a plurality of molecules and the number of molecules included in the plurality of molecules for each constituent unit in each molecule included in the plurality of molecules, and have found that the plurality of molecules may be searched by search based on a cost function including;
(A-1) a negative interaction that is provided to position bits where respective constituent units in one molecule included in the plurality of molecules are adjacent to each other,
(A-2) an original interaction between the molecules that is provided to position bits when the constituent units in the other molecule included in the plurality of molecules are adjacent to each other,
(B-1) a constraint that each of the constituent units in the one molecule exists in one of the position bits prepared for each of the constituent units in the one molecule, and
(B-2) a constraint that one or no constituent unit in any one of the plurality of molecules exists in the position bits at a same position among the position bits prepared for each of the constituent units in the plurality of molecules.

Hereinafter, an example of the technique disclosed herein will be described with reference to the drawings. Processing (an operation) such as the search of the structure formed with the plurality of molecules in the structure search apparatus as an example of the technique disclosed herein may be performed by the structure search unit included in the structure search apparatus.

In one example of the technique disclosed herein, position bits of a number calculated based on the number of constituent units included in a plurality of molecules included in a structure to be searched and the number of molecules included in the plurality of molecules are prepared for each constituent unit in each molecule included in the plurality of molecules.

<Structure to be Searched>

A structure to be searched by using one example of the technique disclosed herein is not particularly limited as long as the structure is formed with a plurality of molecules, and may be appropriately selected depending on a purpose.

A molecule in one example of the technique disclosed herein is not particularly limited and may be appropriately selected depending on the purpose, and examples thereof include a polymer, a protein (peptide), and a low-molecular compound.

The polymer is not particularly limited and may be appropriately selected depending on the purpose, and a plurality of types of polymers may be included in the structure to be searched.

The protein is not particularly limited and may be appropriately selected depending on the purpose, and a protein having a different amino acid sequence may be included in the structure to be searched.

The low-molecular compound is not particularly limited, may be appropriately selected depending on the purpose, and may be, for example, a compound having a molecular weight equal to or lighter than 10000.

The structure to be searched may be, for example, a complex structure of the protein and the low-molecular compound.

<<Constituent Unit>>

The constituent unit in one example of the technique disclosed herein means a unit constituting a molecule included in the structure to be searched. The constituent unit is not particularly limited and may be appropriately selected depending on a kind of the molecule or the like.

When the molecule is the polymer, the constituent unit may be, for example, each of atoms constituting the molecule or a coarse-grained particle (group of atoms) coarse-grained for each of a plurality of atoms.

When the molecule is the protein, the constituent unit may be, for example, a coarse-grained particle coarse-grained for each amino acid residue constituting the protein.

When the molecule is the low-molecular compound, the constituent unit may be, for example, atoms constituting the molecule or a coarse-grained particle coarse-grained for each of a plurality of atoms.

In this way, in one example of the technique disclosed herein, the constituent unit may be a group of atoms or an atom.

<<Position Bit>>

The position bit in one example of the technique disclosed herein means a bit representing a position of the constituent unit constituting the molecule included in the structure to be searched. The position bit, in one example of the technique disclosed herein, means that, for example, when the position bit is “1”, the constituent unit exists in the position bit, and when the position bit is “0”, the constituent unit does not exist in the position bit.

Positions where the position bits are arranged are not particularly limited, may be appropriately selected depending on the purpose, for example, and may be located in a lattice shape or may be irregularly arranged. In one example of the technique disclosed herein, it is preferred that the position bits be located in the lattice shape.

In a case where the position bits are arranged in the lattice shape, a lattice structure is not particularly limited, and may be appropriately selected depending on the purpose, and examples thereof include a planar lattice, a simple cubic lattice, a body-centered cubic lattice, and a face-centered cubic lattice.

In one example of the technique disclosed herein, it is preferable that a periodic boundary condition be imposed on the position bits. The term “periodic boundary condition” means, for example, in a calculation system having a cubic (or rectangular) shape in which the position bits are arranged, a condition that a plurality of virtual calculation systems identical to the calculation system are arranged so as to surround the calculation system. For example, the term “periodic boundary condition” means a condition that situations (states) on two specific boundary surfaces of the calculation system become equal to each other in the calculation system having the cubic (or rectangular) shape in which the position bits are arranged. The two specific boundary surfaces may be, for example, surfaces (or lines) facing each other in the cube (or rectangle).

In the technique disclosed herein, in one aspect, by imposing the periodic boundary condition on the position bits, it is possible to suppress an adverse effect due to presence of a boundary in the calculation system in which the position bits are arranged, so that it is possible to search a structure formed with a plurality of molecules under a condition close to a bulk state. Accordingly, in the technique disclosed herein, in one aspect, it is possible to search the structure formed with the plurality of molecules with higher accuracy, while appropriately considering influence of the boundary in the calculation system.

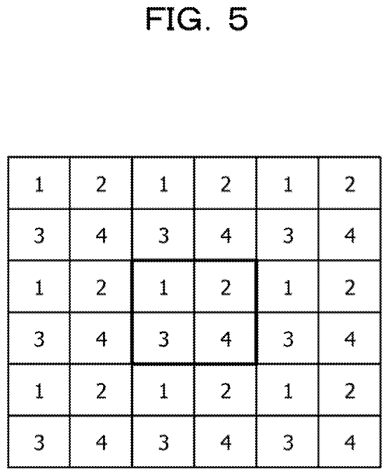

FIG. 5 illustrates an example in which a periodic boundary condition is imposed on a calculation system (central calculation system in FIG. 5) in which four position bits represented by numbers 1 to 4 are arranged, and a virtual calculation system identical to the central calculation system is arranged so as to surround the center calculation system.

For example, the position bit having the number 2 in the calculation system located on a left side of the central calculation system is adjacent to a left side of the position bit having the number 1 in the central calculation system. For this reason, under the periodic boundary condition, for example, a structure is searched under a condition that situations (states) on a boundary on the left side of the position bit having the number 1 and a boundary on a right side of the position bit having the number 2 are equal to each other. For example, under the periodic boundary condition, for example, the structure is searched under the condition that the left side of the position bit having the number 1 and the right side of the position bit having the number 2 may interact with each other.

As described above, in the technique disclosed herein, in one aspect, by imposing the periodic boundary condition on the position bit, it is possible to search the structure formed with a plurality of molecules with higher accuracy, while more appropriately considering an interaction for each constituent unit.

The search of the structure with the periodic boundary condition imposed may be performed, for example, by specifying a combination of the position bits adjacent to the position bit when the periodic boundary condition is imposed for each position bit, and searching the structure based on the specified combination of the position bits.

In an example of the technique disclosed herein, the position bits of the number calculated based on the number of constituent units included in a plurality of molecules included in the structure to be searched and the number of molecules included in the plurality of molecules are prepared for each constituent unit in each molecule included in the plurality of molecules. In the following description, “the number of constituent units included in a plurality of molecules included in the structure to be searched” is referred to as “the number of constituent units”, and “the number of the plurality of molecules included in the structure to be searched” is referred to as “the number of molecules”, in some cases.

The number calculated based on the number of constituent units and the number of molecules may be, for example, a number calculated based on a sum with respect to the number of constituent units and the number of molecules. The number calculated based on the sum with respect to the number of constituent units and the number of molecules may be the sum with respect to the number of constituent units and the number of molecules, or may be larger than the sum with respect to the number of constituent units and the number of molecules. For example, the number calculated based on the number of constituent units and the number of molecules in one example of the technique disclosed herein may be equal to or more than the number of constituent units included in the structure to be searched. For example, in one example of the technique disclosed herein, the position bits of a number larger than a total number of constituent units included in the structure that is formed with the plurality of molecules and that is to be searched may be prepared.

In one example of the technique disclosed herein, the position bits are prepared for each constituent unit in each molecule included in a plurality of molecules. For example, in the example of the technique disclosed herein, the position bits of the number calculated based on the number of constituent units and the number of molecules are prepared for each of all the constituent units included in the structure to be searched.

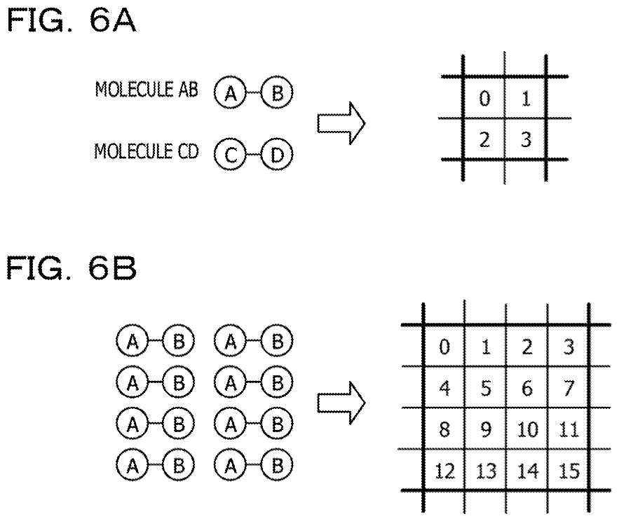

As illustrated in the example of FIG. 6A, in a case where a structure including a molecule AB composed of constituent units A and B and a molecule CD composed of constituent units C and D is searched, description of a case where position bits of a number calculated based on a sum with respect to constituent units and the number of molecules are prepared will be given.

First, in the example of FIG. 6A, each of the molecule AB and the molecule CD is composed of two constituent units. In the example illustrated in FIG. 6A, since the structure to be searched includes two molecules, the molecule AB and the molecule CD, the number of molecules included in the structure to be searched is two.

For example, in the example illustrated in FIG. 6A, since the number of constituent units is two and the number of molecules is two, the sum with respect to the number of constituent units and the number of molecules is four.

Therefore, in the example illustrated in FIG. 6A, when the position bits are prepared for each constituent unit in each molecule, four position bits are prepared for each of four constituent units of the constituent units A to D, so that 16 position bits are prepared in total.

Next, as illustrated in the example of FIG. 6B, in a case where a structure including eight molecules AB composed of the constituent units A and B is searched, a case will be described where position bits of a number calculated based on a sum with respect to the number of constituent units and the number of molecules are prepared.

First, in the example of FIG. 6B, the molecule AB is composed of two constituent units. In the example illustrated in FIG. 6B, since eight molecules AB are included in the structure to be searched, the number of molecules is eight.

For example, in the example illustrated in FIG. 6B, since the number of constituent units is two and the number of molecules is eight, the sum with respect to the number of constituent units and the number of molecules is 16.

In the example illustrated in FIG. 6B, since eight constituent units A and eight constituent units B are included in the structure, 16 constituent units are included in the structure to be searched in total. Therefore, in the example illustrated in FIG. 6B, when the position bits are prepared for each constituent unit in each molecule included in a plurality of molecules, 16 position bits are prepared for each of the 16 constituent units, so that 256 position bits are prepared in total.

In this manner, by preparing the position bits for each constituent unit in each molecule included in the plurality of molecules, the technique disclosed herein may search the structure formed with the plurality of molecules having interactions, in consideration of all positions where the constituent units may exist, in one aspect.

In order to simplify the description, the example of searching the two-dimensional structure has been described, but in an example of the technique disclosed herein, a three-dimensional structure may be searched by arranging position bits in three dimensions.

<Cost Function>

In one example of the technique disclosed herein, a structure formed with a plurality of molecules having interactions is searched based on a cost function including at least four interactions or constraints of the following (A-1) to (B-2).
(A-1) A negative interaction that is provided to position bits where individual constituent units in one molecule included in the plurality of molecules are adjacent to each other
(A-2) An original interaction between molecules that is provided to position bits when constituent units in the other molecule included in the plurality of molecules are adjacent to each other
(B-1) a constraint that each of the constituent units in the one molecule exists in one of the position bits prepared for each of the constituent units in the one molecule, and
(B-2) a constraint that one or no constituent unit in any one of the plurality of molecules exists in the position bits at a same position among the position bits prepared for each of the constituent units in the plurality of molecules.

The cost function including the above (A-1) to (B-2) may be, for example, a sum of a term representing the interaction of (A-1), a term representing the interaction of (A-2), a term representing the constraint of (B-1), and a term representing the constraint of (B-2).

In the technique disclosed herein, an interaction or a constraint other than the above (A-1) to (B-2) may also be included in the cost function. The interaction or the constraint other than (A-1) to (B-2) is not particularly limited, and may be appropriately selected depending on the purpose.

The negative interaction in the above (A-1) is not particularly limited as long as the negative interaction is an interaction which each constituent unit in the one molecule included in the plurality of molecules provides to the position bits adjacent to the constituent unit, and whose magnitude is negative (having a negative sign), and may be appropriately selected depending on the purpose.

The magnitude (strength) of the negative interaction in the above (A-1) is preferably set such that the constituent units in the one molecule are not separated (such that binding in the one molecule is not cut off) when the structure is searched based on the cost function including (A-1) to (B-2), for example. The setting of the negative interaction having such a magnitude that the constituent units in the one molecule are not separated may be actually performed by, for example, searching the structure based on the cost function including (A-1) to (B-2) and tuning the magnitude of the negative interaction.

The position bits where individual constituent units in the one molecule included in the plurality of molecules are adjacent to each other are not limited to only the nearest position bits in which the constituent units are in contact with each other, and may include, for example, position bits other than the nearest position bits in addition to the nearest position bits.

The original interaction between the molecules of the above (A-2) is not particularly limited as long as the original interaction is an interaction which the constituent units in the other molecule included in the plurality of molecules provides to the position bits adjacent to the constituent units, and may be appropriately selected depending on the purpose.

The original interaction means, for example, an electrostatic interaction, an interaction that expresses a van der Waals force, or the like acting between molecules.

The magnitude (strength) of the original interaction in the above (A-2) is, for example, preferably set for each combination of types of the constituent units. By doing so, the technique disclosed herein, in one aspect, may search the structure formed with the plurality of molecules with higher accuracy based on the cost function that includes the original interaction in accordance with properties between the constituent units.

The magnitude of the original interaction in the above (A-2) may be appropriately set based on, for example, the properties of the constituent units, or the like. For example, when the molecule included in the structure to be searched is a polymer or a low-molecular compound, it is preferable to appropriately generate and use a parameter representing the magnitude of the original interaction in each combination of the types of the constituent units, by calculation based on the properties of the constituent units constituting the molecule.

When the molecule included in the structure to be searched is a protein, for example, the parameter representing the magnitude of the original interaction in each combination of the types of the constituent units may be determined by referring to the Miyazawa-Jernigan (MJ) matrix or the like. In a case where the protein includes an unnatural amino acid residue, it is preferred that interaction parameters among the unnatural amino acid residue and other amino acid residues be appropriately created and used.

As the original interaction in the above (A-2), for example, an interaction a and an interaction (to be described below may be different from each other.
Interaction α: An interaction between a constituent unit in one molecule and a constituent unit in the other molecule in a case where the constituent unit in the one molecule is the same as the constituent unit in the other molecule.
Interaction β: An interaction between a constituent unit in one molecule and a constituent unit in the other molecule in a case where the constituent unit in the one molecule and the constituent unit in the other molecule are different from each other.

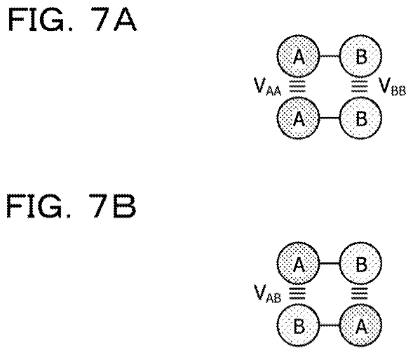

As an example where the interaction α and the interaction β described above are different from each other, description of a case where a structure in which two molecules AB composed of the constituent units A and B are present as illustrated in FIG. 7A and FIG. 7B is searched will be given.

As illustrated in FIG. 7A, for example, in a case where the constituent units A are adjacent to each other and the constituent units B are adjacent to each other in the two molecules AB, an interaction between the constituent units A is defined as V_{AA}, and an interaction between the constituent units B is defined as V_{BB}. Similarly, as illustrated in FIG. 7B, for example, in a case where the constituent unit A and the constituent unit B are adjacent to each other in the two molecules AB, an interaction between the constituent unit A and the constituent unit B is defined as V_{AB}.

In this example, one example of the interaction α is the interactions V_{AA} and V_{BB}, and one example of the interaction β is the interaction V_{AB}. For this reason, in the example illustrated in FIGS. 7A and 7B, for example, the interactions V_{AA} and V_{BB} and the interaction V_{AB} are different from each other, so that the interaction α and the interaction β may be made different from each other.

In the examples illustrated in FIG. 7A and FIG. 7B, the interactions V_{AA} and V_{BB} are the same interaction (having the same magnitude), but the technique disclosed herein is not limited thereto, and interactions V_{AA} and V_{BB} may be different interactions.

The negative interaction in the above (A-1) and the original interaction in the above (A-2) may be expressed as numerical values, for example.

In the negative interaction in the above (A-1) and the original interaction in the above (A-2), it is preferable to satisfy the following inequality [negative interaction<original interaction].

In this way, the technique disclosed herein may more reliably search a structure in which constituent units in one molecule are not separated, in one aspect, so that it is possible to search a structure formed with a plurality of molecules with higher accuracy.

The constraint in the above (B-1) is a constraint to be imposed such that each constituent unit in one molecule exists in one of the position bits prepared for each constituent unit in the one molecule in the position bits prepared for each constituent unit in the one molecule. For example, the constraint in the above (B-1) is a constraint to be imposed such that the constituent unit included in the structure to be searched exists in any one of the position bits prepared for the constituent units so as not to exist in the other position bits.

When the cost function includes the constraint in the above (B-1), in the technique disclosed herein, in one aspect, the constituent units included in the structure to be searched exist one by one, and the structure having no contradiction as a structure formed with a plurality of molecules may be searched. For example, since the cost function includes the constraint in the above (B-1), the technique disclosed herein may suppress variation in the number of constituent units in searching a structures (may fix the number of constituent units) in one aspect, so that it is possible to search the structure that includes the respective constituent units constituting the input molecule one by one.

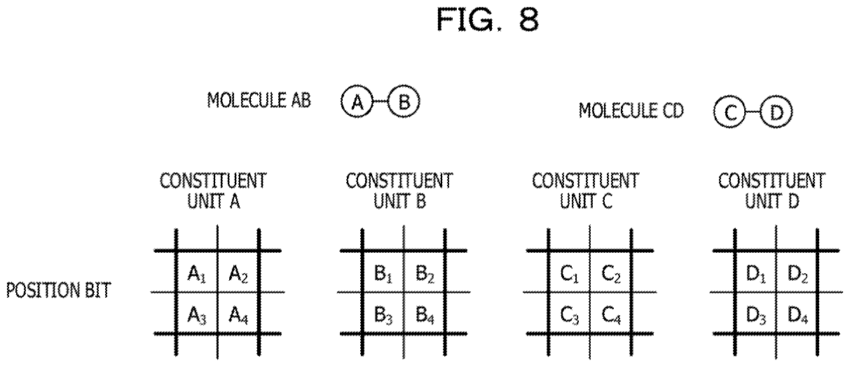

A specific example of the constraint of (B-1) will be described with reference to FIG. 8. In FIG. 8, four position bits prepared for the constituent unit A are defined as respective position bits A₁ to A₄. Similarly to the constituent units B to D, the respective prepared position bits are defined as the position bits B₁ to B₄, the position bits C₁ to C₄, and the position bits D₁ to D₄.

The example illustrated in FIG. 8 is an example in which four position bits are prepared for each of the four constituent units A to D. In the example illustrated in FIG. 8, the position bits prepared for each constituent unit in one molecule correspond to the position bits A₁ to A₄ for the constituent unit A, for example.

As for the position bits A₁ to A₄, a state where each constituent unit in one molecule exists in one of the position bits prepared for each constituent unit in the one molecule means, for example, a state where the constituent unit exists in any one of the position bits A₁ to A₄. For example, the constraint of (B-1) in the example illustrated in FIG. 8 is such a constraint that when a case where the constituent unit exists in the position bit is set to 1, and a case where the constituent unit does not exist in the position bit is set to 0, only one of the position bits A₁ to A₄ for the constituent unit A becomes 1 and the other position bits become 0. By imposing the similar constraint on each of the constituent units B to D, the constraint of (B-1) may be imposed on all the constituent units.

The constraint of the above (B-1) is preferably performed by giving a positive cost to the cost function when the sum of the position bits prepared for the respective constituent units is not 1, when the case where the constituent unit exists in the position bits is set to 1, and the case where the constituent unit does not exist is set to 0, for example. Giving a positive cost to the cost function means, for example, that a value of a term representing the constraint is set to such a value that a value of the cost function becomes large.

The large value of the cost function, for example, may be considered to correspond to a fact that the structure having the value of the cost function is unstable (energy is high).

A fact that the sum of the position bits prepared for the respective constituent units is not 1 corresponds to, for example, a fact that each constituent unit in one molecule does not exist in one of the position bits prepared for each constituent unit in the one molecule (no constituent unit or two or more constituent units exist).

For example, in the constraint of the above (B-1), when the sum of the position bits prepared for the respective constituent units is not 1, giving the positive cost to the cost function makes it possible to increase the value of the cost function in the structure that may have a contradiction as the structure formed with the plurality of molecules. Accordingly, in one aspect of the technique disclosed herein, when the structure is searched by stabilizing the cost function, the respective constituent units included in the structure to be searched exist one by one, and the structure having no contradiction as the structure formed with the plurality of molecules may be searched.

As the constraint of the above (B-1), a specific example of the term representing the constraint of the above (B-1) constraint in the case where the positive cost is given to the cost function when the sum of the position bits prepared for the respective constituent units is not 1 will be described with reference to FIG. 8.

In the example illustrated in FIG. 8, for example, as for the position bits A₁ to A₄ for the constituent unit A, when p is defined as a coefficient (positive number), p (A₁+A₂+A₃+A₄−1)² may be used as the term representing the constraint of the above (B-1). In the above term, when only one position bit of the position bits A₁ to A₄ becomes 1 and the other position bits become 0, a value of the term becomes 0, but in the other cases, the value of the term becomes a positive value. For example, the above term is a term that gives the positive cost to the cost function when the sum of the position bits A₁ to A₄ is not 1. By using the similar term for the constituent units B to D, as for all the constituent units, when the sum of the position bits is not 1, the positive cost may be given to the cost function.

The constraint of the above (B-2) is a constraint to be imposed such that one or no constituent unit in any one of the plurality of molecules exists in the position bits at the same position bit among the position bits prepared for each constituent unit in the plurality of molecule. For example, the constraint of the above (B-2) is a constraint to be imposed such that the different constituent units do not exist so as to overlap with each other in the position bits at the same position.

Because the cost function includes the constraint of the above (B-2), in one aspect of the technique disclosed herein, different constituent units do not exist at the same position, and the structure having no contradiction as the structure formed with the plurality of molecules may be searched. For example, since the cost function includes the constraint of the above (B-2), the technique disclosed herein may be used to search the structure in which the respective constituent units are located at different positions, because overlap of different constituent units in searching the structure may be suppressed, in one aspect.

A specific example of the constraint of (B-2) will be described with reference to FIG. 8. The example illustrated in FIG. 8 is an example in which four position bits are prepared for each of four constituent units of the constituent units A to D. In FIG. 8, the position bits at the same position among the position bits prepared for each constituent unit in the plurality of molecules correspond to, for example, the position bit A₁, of the constituent unit A, the position bit B₁ of the constituent unit B, the position bit C₁ of the constituent unit C, and the position bit D₁ of the constituent unit D.

In the position bits A₁, B₁, C₁, and D₁, the state in which one or no constituent unit exists in the position bits at the same position means, for example, a state in which a constituent unit exists in any one of the position bits A₁, B₁, C₁, and D₁, or no constituent unit exists. For example, the constraint of (B-2) in the example illustrated in FIG. 8 is a constraint that the sum of the position bits A₁, B₁, C₁, and D₁ does not become equal to or larger than two, when the case where the constituent unit exists in the position bit is set to 1 and the case where the constituent unit does not exist in the position bit is set to 0. By imposing the similar constraint on the position bits A₂ to D₂, the position bits A₃ to D₃, and the position bits A₄ to D₄, it is possible to impose the constraint of (B-2) on all of the constituent units.

The constraint of the above (B-2) is preferably imposed by giving a positive cost to the cost function when the sum of the position bits at the same position is not 0 or 1 among the position bits prepared for each constituent unit, for example. As in the case of (B-1), the case where the constituent unit exists in the position bit is set to 1, and the case where the constituent unit does not exist in the position bit is set to 0.

The fact that the sum of the position bits at the same position is not 0 or 1 among the position bits corresponds to, for example, a fact that the constituent units different from each other (two or more) exist so as to overlap in the position bit at the same position.

For example, when the sum of the position bits at the same position is not 0 or 1 among the position bits, giving the positive cost to the cost function makes it possible to increase the value of the cost function in the structure that may have a contradiction as the structure formed with the plurality of molecules. Accordingly, in one aspect of the technique disclosed herein, when the structure is searched by stabilizing the cost function, the constituent units different from each other do not exist so as to overlap at the same position, and the structure having no contradiction as the structure formed with the plurality of molecules may be searched.

As the constraint of the above (B-2), a specific example of the term representing the constraint of the above (B-2) in the case where when the sum of the position bits at the same position is not 0 or 1 among the position bits, the cost function is given the positive cost will be described with reference to FIG. 8.

In the example illustrated in FIG. 8, for example, as for the position bits A₁, B₁, C₁, and D₁, when p is defined as a coefficient (positive number), p (A₁+B₁+C₁+D₁−1)² may be used as the term representing the constraint of the above (B-2). In the above term, when only one of the position bits A₁, B₁, C₁, and D₁ is 1 and the other position bits are 0, the value of the term becomes 0, but in the other cases, the value of the term becomes a positive value. For example, the above term is a term which gives the positive cost to the cost function when the sum of the position bits A₁, B₁, C₁, and D₁ is not 1. By using the similar term for the position bits A₂ to D₂, the position bits A₃ to D₃, and the position bits A₄ to D₄, it is possible to give the positive cost to the cost function for each of all the constituent units when the sum of the position bits is not 1.

In the above example, the example has been described in which when the sum of the position bits at the same position is not 1, the cost function is given the positive cost, but as described above, even when the sum of the position bits at the same position is not 0, the positive cost may be given to the cost function. In this case, for example, as for the position bits A₁, B₁, C₁, and D₁, when p is defined as a coefficient (positive number), p (A₁+B₁+C₁+D₁−1) (A₁+B₁+C₁+D₁) may be used as the term representing the constraint of the above (B-2). In the above term, when all of the position bits A₁, B₁, C₁, and D₁ are 1 or 0, the value of the term becomes 0, but in the other cases, the value of the term becomes a positive value.

Examples of the case where the sum of the position bits at the same position becomes 0 among the position bits include, for example, a case where the position bits of a number larger than a total number of constituent units included in the structure that is formed with the plurality of molecules and that is to be searched are prepared, and the like.

In this manner, in one example of the technique disclosed herein, by performing the search based on the cost function including the four interactions or constraints of the above (A-1) to (B-2), it is possible to correctly search the structure formed with the plurality of molecules.

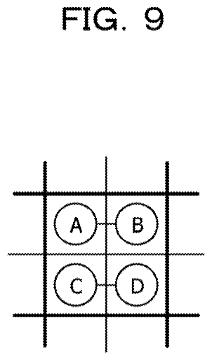

In terms of the example illustrated in FIG. 6, when the structure is searched by using one example of the technique disclosed herein, a structure as illustrated in FIG. 9 may be obtained, for example. In the example illustrated in FIG. 9, the structure is searched in which the constituent units in one molecule are not separated from each other, and the constituent units do not overlap with each other, and are arranged one by one in each of the position bits.

<<Specific Example of Cost Function>>

The cost function in one example of the technique disclosed herein is not particularly limited as long as the cost function includes the four interactions or constraints of (A-1) to (B-2), and may be appropriately selected depending on the purpose, but it is preferable to use, for example, a cost function of the following Equation (1).

$$E = {\sum }_{{N}_{i} = 0}^{N - 1} {\sum }_{〈 n , {n}^{′} 〉}^{ } {\sum }_{{i}_{p} = 0}^{{N}_{p} - 1} {vx}_{m} {x}_{{m}^{′}} + {\sum }_{〈 {N}_{i} , {N}_{i}^{′} 〉} {\sum }_{n = 0}^{{n}_{{N}_{i}} - 1} {\sum }_{{n}^{′} = 1}^{{n}_{{N}_{i}^{′}} - 1} {E}_{pair} {x}_{m} {x}_{{m}^{′}} + {p}_{1} {\sum }_{m = 0}^{M - 1} {( {\sum }_{i = 0}^{t - 1} {x}_{m} - 1 )}^{2} + {p}_{2} {\sum }_{i = 0}^{t - 1} {( {\sum }_{m = 0}^{M - 1} {x}_{m} - 1 )}^{2} \qquad ( 1 )$$

In the above Equation (1),

E is the cost function.

N is the number of molecules included in the structure to be searched, and Nᵢ is the number of the molecule.

n is the number of the constituent unit in one molecule.

Nₚ is the number of adjacent position bits in the position bits prepared for each constituent unit in each molecule, and iₚ is the number of an adjacent position bit in the position bits prepared for each constituent unit in each molecule.

v is a numerical value representing the magnitude of the negative interaction in (B-1).

xₘ is a binary variable representing that the position bit at the m-th position is 0 or 1.

n_{Ni} is the number of constituent units in one molecule.

Eₚₐᵢᵣ is a numerical value representing the magnitude of the original interaction in (B-2).

p₁ and p₂ are positive numbers.

M is a total number of the constituent units included in the structure to be searched.

t is the number of the position bits prepared for each constituent unit in each molecule, and i is the number of a position bit prepared for each constituent unit in each molecule.

In the above Equation (1), a notation represented by, for example, <i, j> means a pair of i and j. In the above Equation (1), i={0, 1, 2, . . . t−1}, Nᵢ={0, 1, 2, . . . N−1}, n={0, 1, 2, . . . n_{Ni}−1}, and iₚ={0, 1, 2, . . . Nₚ−1} are satisfied. In the above Equation (1), a total number of the prepared position bits is represented by tM.

In addition, in the above Equation (1), M satisfies the following equation.

$$M = {\sum }_{{N}_{i} = 1}^{N} {n}_{{N}_{i}}$$

In the above Equation (1), m meaning a serial number of the prepared position bits satisfies the following equation.

$$m = i + tn + t {\sum }_{{N}_{j} = 1}^{{N}_{i - 1}} {n}_{{N}_{j}} = 0 , , , tM - 1$$

Parameters in the above Equation (1) may be appropriately set based on information of the molecules and the constituent units included in the structure to be searched. For example, as for v, p₁, and p₂, it is preferable to actually perform the search of the structure based on the above Equation (1) to tune the numerical values.

In one example of the technique disclosed herein, a first term on a right side in the above Equation (1) corresponds to the negative interaction in (A-1), a second term on the right side corresponds to the original interaction in (A-2), a third term on the right side corresponds to the constraint of (B-1), and a fourth term on the right side corresponds to the constraint of (B-2) respectively.

The first term on the right side of the above Equation (1) corresponding to the negative interaction in (A-1) is a term representing the sum of the magnitudes of the negative interactions among the position bits adjacent to each other in the position bits prepared for each individual constituent unit in each molecule.

Since v in the first term on the right side of the above Equation (1) is a negative number in many cases, when xₘ and xₘ′ are 1, the first term on the right side becomes a negative number having a larger absolute value, and the value of the cost function becomes small. The fact that the value of the cost function is small may be considered to correspond to, for example, a fact that the structure having the value of the cost function is stable (energy is low).

The second term on the right side of the above Equation (1) corresponding to the original interaction in (A-2) is a term representing the sum of the magnitudes of the original interactions among the position bits where the constituent units in the different molecules are adjacent to each other.

Since Eₚₐᵢᵣ in the second term on the right side of the above Equation (1) is a negative number in many cases, when xₘ and xₘ′ are 1, the second term on the right side becomes a negative number having a larger absolute value, and the value of the cost function becomes small.

The third term on the right side of the above Equation (1) corresponding to the constraint of (B-1) is a term representing a penalty which gives a positive cost to the cost function when the sum of the position bits prepared for the respective constituent units is not 1 (the value of the cost function is increased).

Since p₁ in the third term on the right side of the above Equation (1) is a positive number, when the sum of each xₘ for the position bits prepared for the respective constituent units is not 1, the third term on the right side becomes a larger positive number, and the value of the cost function becomes large.

The fourth term on the right side of the above the above Equation (1) corresponding to the constraint of (B-2) is a term representing a penalty which gives a positive cost to the cost function when the sum of the position bits at the same position is not 1 among the position bits prepared for the respective constituent units.

Since p₂ in the fourth term on the right side of the above Equation (1) is a positive number, when the sum of the position bits xₘ at the same position among the position bits prepared for the respective constituent units is not 1, the fourth term on the right side becomes a larger positive number, and the value of the cost function becomes large.

The fourth term on the right side of the above Equation (1) is a term which gives a positive cost to the cost function when the sum of the position bits at the same position is not 1, but the fourth term on the right side of Equation (1) may be modified so as to give a positive cost even when the sum of the position bits at the same position is not 0.

In one example of the technique disclosed herein, it is preferable to search the structure formed with the plurality of molecules based on a cost function obtained by converting the above Equation (1) into an Ising model represented by the following Equation (2).

$$E = - {\sum }_{i , j = 0}^{ } {w}_{ij} {x}_{i} {x}_{j} - {\sum }_{i = 0}^{ } {b}_{i} {x}_{i} \qquad ( 2 )$$

In the above Equation (2),

wᵢⱼ is a coefficient for weighting between the position bit at an i-th position and the position bit at a j-th position.

bᵢ is a numerical value representing a bias for the position bit at the i-th position.

xᵢ is a binary variable representing that the position bit at the i-th position is 0 or 1, and xⱼ is a binary variable representing that the position bit at the j-th position is 0 or 1.

wᵢⱼ may be obtained, for example, by extracting v, Eₚₐᵢᵣ, p₁, and p₂ in the above Equation (1) for each combination of xᵢ and xⱼ, and is a matrix in many cases.

A first term on a right side of the above Equation (2) represents an integration of products of states and weight values of two circuits without missing or redundantly counting for all combinations of two circuits selectable from all circuits.

A second term on the right side of the above Equation (2) represents an integration of products of the respective bias values and states of all the circuits. For example, by extracting the parameters of the above Equation (1) and obtaining wᵢⱼ and bᵢ, the above Equation (1) may be converted into the Ising model expressed by the above Equation (2).

The stabilization of a cost function (Hamiltonian) expressed by the Ising model equation of a quadratic constrained binary optimization (QUBO) format, as in the above Equation (2) may be performed in a short time by performing an annealing method (annealing) using an annealing machine or the like.

Therefore, according to the technique disclosed herein, in one aspect, it is possible to search the structure with the plurality of molecules by the annealing method using the annealing machine or the like by using the above Equation (2), so that the structure may be searched in a shorter time. For example, in one aspect of the technique disclosed herein, the structure may be searched in a shorter time by stabilizing the cost function by the annealing method. The annealing method will be described in detail later.

In one example of the technique disclosed herein, it is preferable to search the structure formed with the plurality of molecules having interactions by minimizing the cost function by the annealing method. In this manner, the technique disclosed herein may search a most stable structure in which the cost function is minimum in a short time, in one aspect.

The most stable structure in which the cost function is minimum may be considered to correspond to a structure formed with a plurality of molecules at absolute zero (0 K), for example.

In one example of the technique disclosed herein, it is also preferable to search the structure formed with the plurality of molecules at one temperature by repeating decreasing a temperature from a temperature higher than the one temperature to the one temperature a plurality of times to perform averaging, while changing the position bit by using a random number. As a method for changing the position bit using the random number, a metropolis method may be used, for example.

The one temperature is not particularly limited and may be appropriately selected depending on the purpose, and for example, a temperature other than absolute zero (finite temperature) may be used. Since the structure formed with the plurality of molecules at a finite temperature may not be uniquely determined due to the influence of fluctuation caused by the temperature or the like, it is preferable to repeat decreasing temperature from the temperature higher than the one temperature to the one temperature the plurality of times to perform averaging, while changing the position bit by using the random number.

Thus, the technique disclosed herein, in one aspect, may search the structure formed with the plurality of molecules having interactions at a desired temperature (one temperature).

For example, by searching the structure at a plurality of temperatures by changing the one temperature, it is possible to analyze transition of the structure due to a change in temperature. Accordingly, in the technique disclosed herein, it is possible to observe a state in which an energy phase gently changes from a regular state to an irregular state at a transition temperature as a border, in one aspect.

In one example of the technique disclosed herein, it is also preferable to search the structure formed with the plurality of molecules at the one temperature by performing a calculation in which the one temperature is held for a certain period of time by a replica exchange method to perform averaging.

The replica exchange method is a method in which systems (replicas) which do not interact with each other and have different temperatures are prepared, and the temperatures of the respective systems are exchanged under predetermined conditions.

According to one aspect of the technique disclosed herein, the structure formed with the plurality of molecules having interactions at a desired temperature (one temperature) may be searched by performing a calculation in which the one temperature is held for a certain period of time by the replica exchange method to perform averaging.

An example of the technique disclosed herein will be described in more detail with reference to a configuration example of the apparatus and a flowchart.

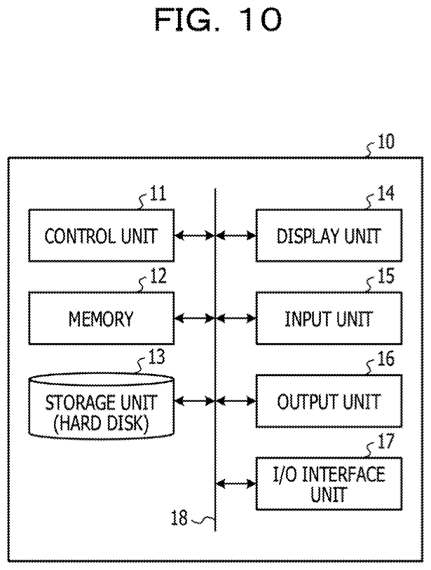

FIG. 10 illustrates a hardware configuration example of a structure search apparatus disclosed herein. In a structure search apparatus 10, for example, a control unit 11, a memory 12, a storage unit 13, a display unit 14, an input unit 15, an output unit 16, and an I/O interface unit 17 are coupled via a system bus 18.

The control unit 11 performs operations (four arithmetic operations, comparison operation, operations for the annealing method, and the like), operation control of hardware and software, and the like.

The control unit 11 is not particularly limited, may be appropriately selected depending on the purpose, and may be, for example, a central processing unit (CPU) or an optimization apparatus to be used in the annealing method to be described later, and may be a combination thereof.

A structure search unit in the structure search apparatus disclosed herein may be implemented by, for example, the control unit 11.

The memory 12 is a memory such as a random-access memory (RAM), a read-only memory (ROM), or the like. The RAM stores an operating system (OS), an application program, and the like read from the ROM and the storage unit 13, and functions as a main memory and a work area of the control unit 11.

The storage unit 13 is a device for storing various programs and data, and is a hard disk, for example. The storage unit 13 stores a program to be executed by the control unit 11, data to be used for execution of the program, the OS, and the like.

A structure search program disclosed herein is stored in the storage unit 13, is loaded into the RAM (main memory) of the memory 12, and is executed by the control unit 11.

The display unit 14 is a display device, and is, for example, a display device such as a cathode-ray tube (CRT) monitor, or a liquid crystal panel.

The input unit 15 is an input device for various data, and is, for example, a keyboard, a pointing device (for example, a mouse, or the like), or the like.

The output unit 16 is an output device for various data, and is, for example, a printer, or the like.

The I/O interface unit 17 is an interface for coupling various external devices. The I/O interface unit 17 allows input/output of data such as a compact disc read-only memory (CD-ROM), a digital versatile disk read-only memory (DVD-ROM), a magneto-optical (MO) disk, and a Universal Serial Bus (USB) memory [flash drive], for example.

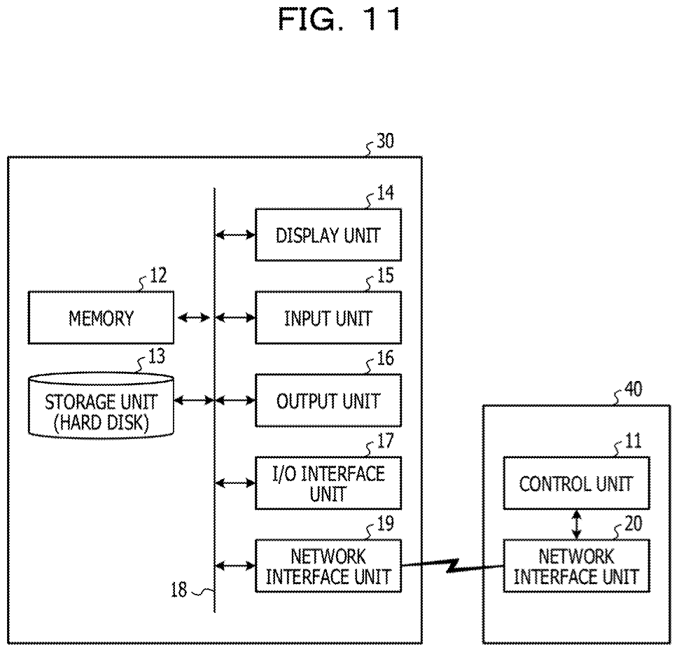

FIG. 11 illustrates another hardware configuration example of the structure search apparatus disclosed herein.

The example illustrated in FIG. 11 is an example in which the structure search apparatus is a cloud type, and the control unit 11 is independent from the storage unit 13 and the like. In the example illustrated in FIG. 11, a computer 30 in which the storage unit 13 and the like are stored, and a computer 40 in which the control unit 11 is stored are coupled via network interface units 19 and 20.

The network interface units 19 and 20 are hardware configured to perform communication by using the Internet.

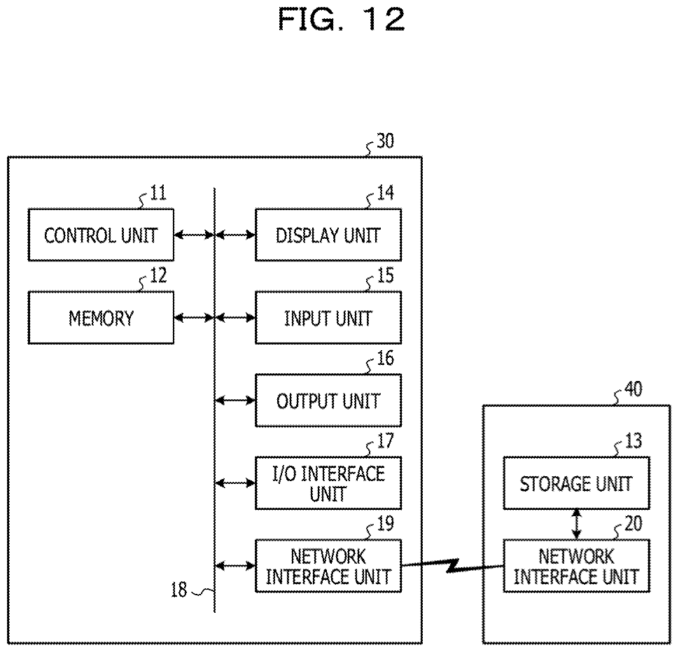

FIG. 12 illustrates another hardware configuration example of the structure search apparatus disclosed herein. The example illustrated in FIG. 12 is an example in which the structure search apparatus is a cloud type, and the control unit 11 is independent from the storage unit 13 and the like. In the example illustrated in FIG. 12, the computer 30 in which the control unit 11 and the like are stored, and the computer 40 in which the storage unit 13 is stored are coupled via the network interface units 19 and 20.

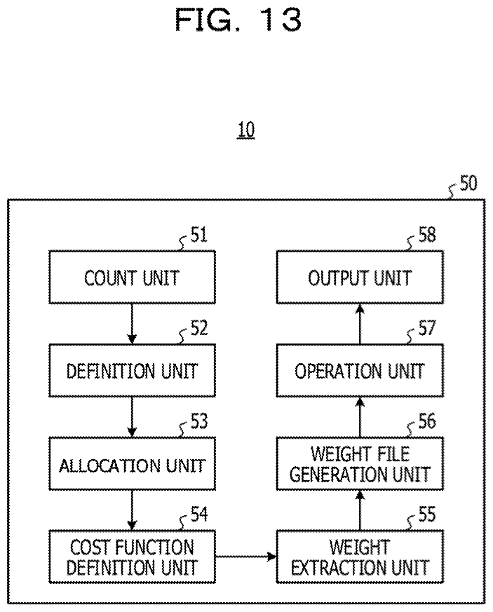

FIG. 13 illustrates a functional configuration example of the structure search apparatus disclosed herein. The structure search apparatus 10 illustrated in FIG. 13 includes a structure search unit 50, and the structure search unit 50 includes a count unit 51, a definition unit 52, an allocation unit 53, a cost function definition unit 54, a weight extraction unit 55, a weight file generation unit 56, an operation unit 57, and a result output unit 58.

The count unit 51 counts the number of molecules and the number of constituent units constituting each molecule in the input structure formed with the plurality of molecules.

The definition unit 52 defines the number of position bits to be prepared for each constituent unit in each molecule included in the structure to be searched, based on the number of constituent units of each molecule and the number of molecules that are counted. The definition unit 52 defines the number of position bits in consideration of dimensions and periodicity of the structure to be searched (for example, whether or not to impose a periodic boundary condition).

The allocation unit 53 allocates (prepares) the position bits of the number defined by the definition unit 52 to the respective constituent units in each molecule included in the structure to be searched. For example, the allocation unit 53 allocates spatial information to each of bits X₁ to Xₙ. The allocation unit 53 specifies a combination of adjacent position bits in consideration of the periodicity of the structure to be searched.

The cost function definition unit 54 defines a cost function including the four interactions or constraints of (A-1) to (B-2). The cost function definition unit 54 defines the cost function represented by the above Equation (1).

The weight extraction unit 55 extracts the parameters (v, Eₚₐᵢᵣ, p₁, and p₂) of the above Equation (1) defined by the cost function definition unit 54.

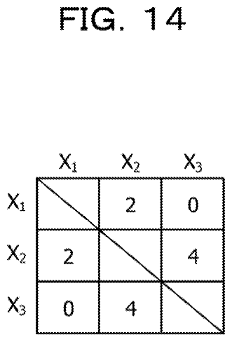

The weight file generation unit 56 generates a weight file corresponding to the extracted weight coefficient. The weight file is a matrix, for example, and in a case of 2X₁X₂+4X₂X₃, the weight file is a file of the matrix as illustrated in FIG. 14. For example, the weight file generation unit 56 specifies wᵢⱼ and bᵢ in the above Equation (2) by using the extracted parameters, and converts the above Equation (1) into an equation of the Ising model expressed by the above Equation (2).

The operation unit 57 stabilizes the equation of the Ising model expressed by the above Equation (2) by the annealing method, thereby searching the structure formed with the plurality of molecules.

The result output unit 58 outputs a search result of the structure by the operation unit 57. The result may be output as a three-dimensional structure diagram of the molecules, or may be output as coordinate information of the constituent units constituting the molecules. The result output from the result output unit 58 may be displayed, for example, by the output unit 16.

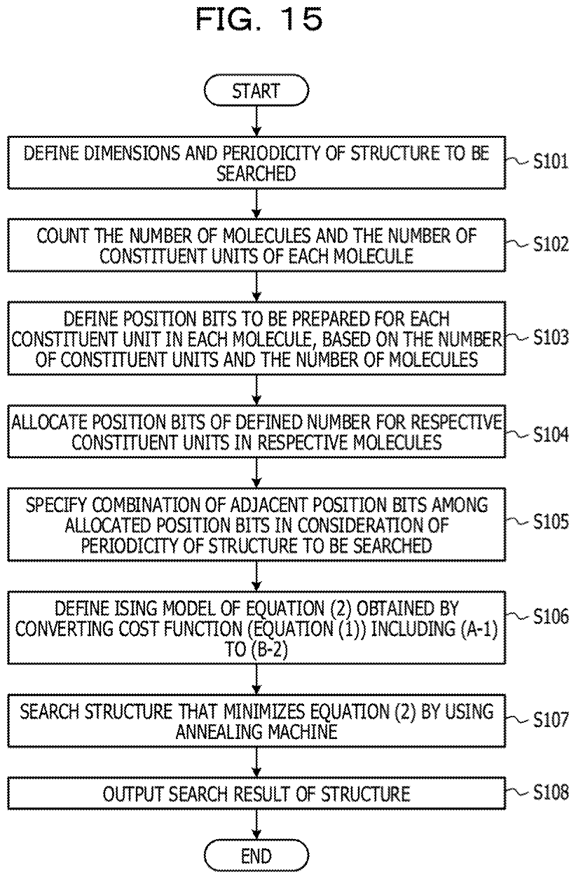

FIG. 15 illustrates an example of a flowchart in searching the structure formed with the plurality of molecules by using an example of the technique disclosed herein.

First, the control unit 11 defines the dimensions (two dimensions or three dimensions) and the periodicity (for example, whether to impose the periodic boundary condition or not) of the structure to be searched (S101). In S101, for example, the structure search apparatus 10 may specify the dimensions and the periodicity by receiving an input from a user, or based on input structure data.

Subsequently, the control unit 11 counts the number of molecules and the number of constituent units constituting each molecule in the input structure formed with the plurality of molecules (S102).

Next, based on the number of constituent units of each molecule and the number of molecules that are counted, the control unit 11 defines the number of position bits to be prepared for each constituent unit in each molecule included in the structure to be searched (S103). When the number of position bits to be prepared for each constituent unit is defined in S103, the dimensions and the periodicity of the structure to be searched are considered.

Next, the control unit 11 allocates the position bits of the number defined in S103 to the respective constituent units in the respective molecule included in the structure to be searched (S104).

The control unit 11 specifies the combination of adjacent position bits in consideration of the periodicity of the structure to be searched for the position bits allocated (prepared) in S104 (S105).

Subsequently, the control unit 11 defines an equation of the Ising model expressed by the above Equation (2) which is obtained by converting the above Equation (1) which is the cost function including the four interactions or constraints of (A-1) to (B-2) (S106).

Next, the control unit 11 minimizes the equation of the Ising model expressed by the above Equation (2) by the annealing method using an annealing machine, thereby searching for the most stable structure in which the cost function is minimum (S107).

The annealing machine is not particularly limited as long as it is a computer employing an annealing method for performing ground state search on an energy function represented by the Ising model, and may be appropriately selected depending on the purpose. Examples of the annealing machine include a quantum annealing machine, a semiconductor annealing machine using a semiconductor technique, and a machine for performing simulated annealing to be executed by software using a CPU or a graphics processing unit (GPU). As the annealing machine, for example, a Digital Annealer (registered trademark) may be used.

In S108, a calculation result is output. The result may be output as a three-dimensional structure diagram of the molecules, or may be output as coordinate information of the constituent units constituting the molecules.

In this way, in the example of the flowchart illustrated in FIG. 15, it is possible to output the search result of the most stable structure in which the cost function is minimum.

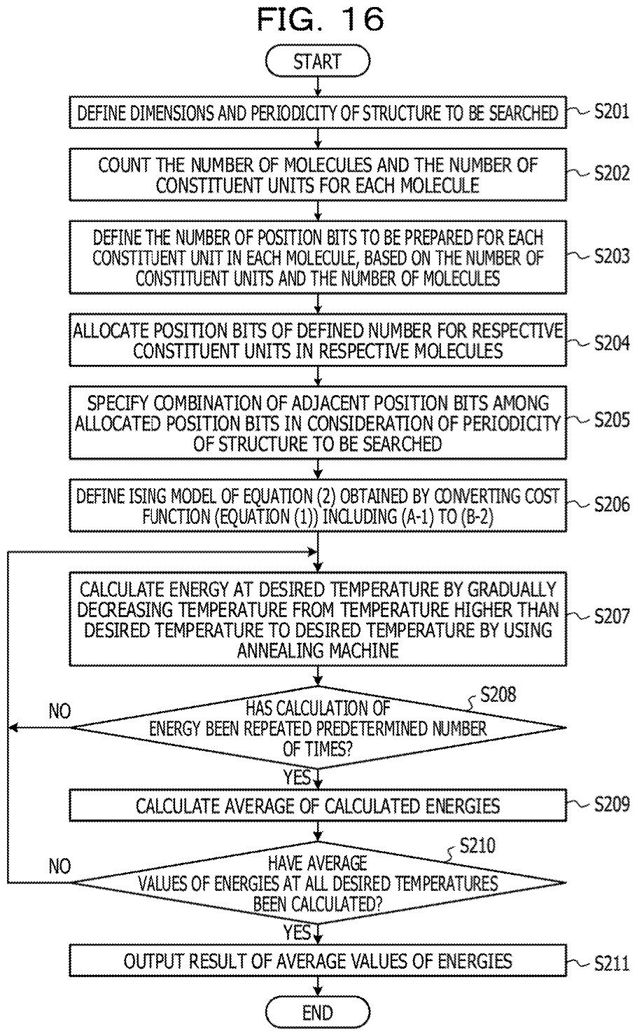

FIG. 16 illustrates an example of a flowchart when the structure at a plurality of desired temperatures (one temperature) is searched and an energy of the structure is calculated, by using an example of the technique disclosed herein.

In FIG. 16, since processes of S201 to S206 are similar to processes of S101 to S106 in FIG. 15, description thereof will be omitted.

In S207, the control unit 11 causes a temperature from a temperature higher than a desired temperature to the desired temperature to decrease, while changing the position bit by using a random number for the equation of the Ising model expressed by the above Equation (2), by using the annealing machine. In S207, an energy (a value of the cost function) at the desired temperature is calculated.

Subsequently, in step S208, the control unit 11 determines whether the calculation of the energy in S207 has been repeated a predetermined number of times or not. In a case where the control unit 11 determines that the calculation of the energy in S207 has been repeated the predetermined number of times, the control unit 11 moves the processing to S209. On the other hand, in a case where the control unit 11 determines that the calculation of the energy in S207 has not been repeated the predetermined number of times yet, the control unit 11 returns the processing to S207.

In S209, the control unit 11 obtains an average of the energies calculated in S207.

Next, in S210, the control unit 11 determines whether or not the averages of the energies have been calculated for all the desired temperatures in S209. In a case where the control unit 11 determines that the energies of the averages have not been calculated for all desired temperatures, the control unit 11 changes the desired temperature and moves the processing to S207. On the other hand, in a case where the control unit 11 determines that the energies of the averages have been calculated for all the desired temperatures, the control unit 11 moves the processing to S211.

In S211, the calculation result is output. The result may be output in a form of, for example, a graph in which a vertical axis represents the energy (value of the cost function) and a horizontal axis represents the temperature.

In this manner, in the example of the flowchart illustrated in FIG. 16, it is possible to output the calculation result of the energies of the structure when the structure at the plurality of desired temperatures (one temperature) is searched.

An example of the annealing method and the annealing machine will be described below.

The annealing method is a method of stochastically obtaining a solution by using a random number value or a superposition of quantum bits. Hereinafter, a problem of minimizing a value of an evaluation function to be optimized will be described as an example, and the value of the evaluation function will be referred to as energy. When the value of the evaluation function is maximized, a sign of the evaluation function may be changed.

First, starting with an initial state in which one discrete value is assigned to each variable, based on a current state (a combination of values of variables), a state close to the current state (for example, a state in which only one of the variables has been changed) is selected, and this state transition is examined. A change in energy associated with the state transition is calculated, and it is stochastically determined whether to adopt the state transition and change the current state or to maintain the original state without adopting the state transition, according to the calculated value. When setting an adoption probability of a state transition that results in a drop in the energy to be larger than that of a state transition that results in a rise in the energy, state changes occur in a direction in which the energy drops on average, and thus it is possible to expect that the state is transitioned to a more suitable state with the lapse of time. Therefore, an approximate solution that possibly results in energy close to an optimal solution or optimal value may be finally obtained.

When a state transition that results in a drop in the energy in a deterministic way is adopted and a state transition that results in a rise in the energy is not adopted, the change in energy broadly monotonically decreases over time, but once a local solution is reached, no further change may occur. Since an extraordinarily large number of local solutions exist in a discrete optimization problem as described above, the state is stuck at a local solution that is not very close to an optimal value, in many cases. Therefore, in solving a discrete optimization problem, it is important to determine whether or not to adopt the state stochastically.

In the annealing method, it has been proved that the state reaches the optimal solution at a limit of infinite time (the number of iterations) as long as the adoption (acceptance) probability of the state transition is determined as follows.

Hereinafter, a method for determining an optimal solution using the annealing method will be described in order.

For an energy change (energy decrease) value (−ΔE) associated with a state transition, an acceptance probability p of the state transition is determined by any of the following functions f( ).

$$\begin{matrix}p ( Δ E , T ) = f ( - Δ E \text{/} T ) & ( 3 \text{-} 1 ) \\ \begin{matrix}{f}_{metro} ( x ) = min ( 1 , {e}^{x} ) & ( Metropolis Method )\end{matrix} & ( 3 \text{-} 2 ) \\ \begin{matrix}{f}_{Gibbs} ( x ) = \frac{1}{1 + {e}^{- x}} & ( Gibbs Method )\end{matrix} & ( 3 \text{-} 3 )\end{matrix}$$

T is a parameter called a temperature value, and for example, may be changed as follows.

The temperature value T is logarithmically reduced with respect to the number of iterations t as expressed by the following equation.

$$T = \frac{{T}_{0} log ( c )}{log ( t + c )} \qquad ( 4 )$$

To represents an initial temperature value and it is desirable that a sufficiently large value be set in accordance with the problem.

In a case of using the acceptance probability expressed by Equation (3-1), when a steady state is reached after the sufficient number of iterations, an occupation probability of each state follows a Boltzmann distribution in a thermal equilibrium state in thermodynamics.

Since the occupation probability of a lower-energy state increases when the temperature is gradually decreased from the high initial temperature, a low-energy state is supposed to be obtained when the temperature sufficiently decreases. This method is referred to as an annealing method (or simulated annealing method) because this behavior resembles state change in annealing a material. The stochastic occurrence of a state transition that results in a rise in the energy corresponds to thermal excitation in physics.

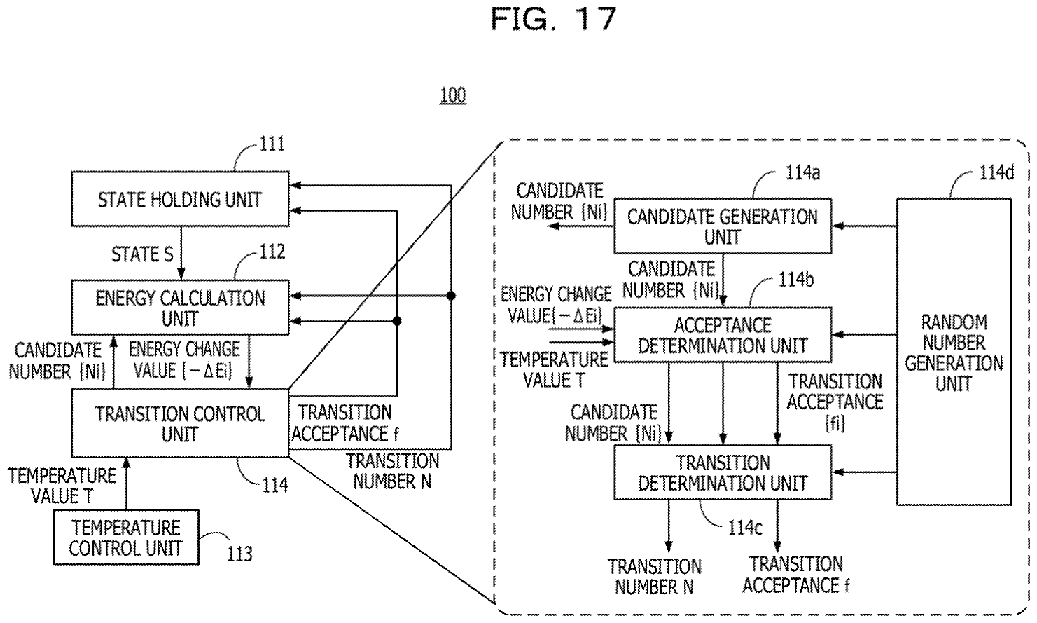

FIG. 17 illustrates an example of a functional configuration of an optimization apparatus (control unit 11) that performs the annealing method. While a case where a plurality of candidates for the state transition is generated will be also described in the following description, the transition candidates are generated one by one in the basic annealing method.

An optimization apparatus 100 includes a state holding unit 111 configured to hold a current state S (values of a plurality of state variables). The optimization apparatus 100 also includes an energy calculation unit 112 configured to calculate energy change values {−ΔEi} of the respective state transitions in a case where the state transition occurs from the current state S as a result of change in any of the values of the plurality of state variables. The optimization apparatus 100 further includes a temperature control unit 113 configured to control a temperature value T and a transition control unit 114 configured to control state changes.

The transition control unit 114 stochastically determines whether or not any one of a plurality of state transitions is accepted, depending on a relative relationship between the energy change values {−ΔEi} and thermal excitation energy based on the temperature value T, the energy change values {−ΔEi}, and the random number value.

The transition control unit 114 includes a candidate generation unit 114a for generating candidates for a state transition, and an acceptance determination unit 114b for stochastically determining whether or not the state transition is accepted for each candidate from the energy change value {−ΔEi} of the candidate and the temperature value T. The transition control unit 114 includes a transition determination unit 114c for determining a candidate to be adopted from the accepted candidates, and a random number generation unit 114d for generating a random variable.

The operation in one iteration in the optimization apparatus 100 is as follows.

First, the candidate generation unit 114a generates one or a plurality of candidates (candidate numbers {Ni}) for the state transition from the current state S held by the state holding unit 111 to the next state. Next, the energy calculation unit 112 calculates the energy change value {−ΔEi} for each of state transitions as the candidates, by using the current state S and the candidates for the state transition. The acceptance determination unit 114b uses the temperature value T generated in the temperature control unit 113 and a random variable (random number value) generated by the random number generation unit 114d, and accepts the state transition with the acceptance probability expressed by the above Equation (3-1) according to the energy change value {−ΔEi} of each of the state transitions.

The acceptance determination unit 114b outputs the acceptances {fi} of the respective state transitions. In a case where a plurality of state transitions is accepted, the transition determination unit 114c randomly selects one thereof by using a random number value. The transition determination unit 114c then outputs a transition number N of the selected state transition, and a transition acceptance f. In a case where there is an accepted state transition, the values of the state variables stored in the state holding unit 111 are updated according to the adopted state transition.

Starting with the initial state, the above iteration processes are repeated while causing the temperature control unit 113 to lower the temperature value, and the operation ends when a certain number of iterations is reached, or when an end determination condition, for example, a condition that the energy becomes lower than a predetermined value, is satisfied. A solution output by the optimization apparatus 100 is the state corresponding to the end of the operation.

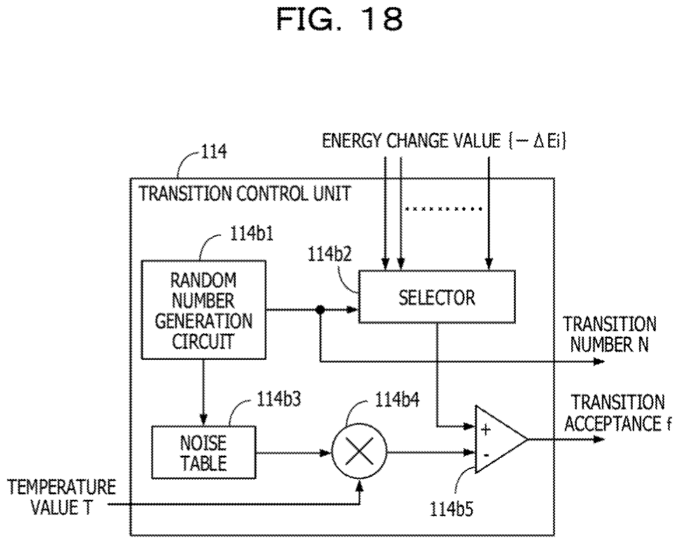

FIG. 18 is a block diagram of a transition control unit in a normal annealing method that generates candidates one by one, and especially, a block diagram of a configuration example of an operation portion to be used for the acceptance determination unit at a circuit level.

The transition control unit 114 includes a random number generation circuit 114b1, a selector 114b2, a noise table 114b3, a multiplier 114b4, and a comparator 114b5.

Of all the energy change values {−ΔEi} calculated for the candidates of the respective state transitions, the selector 114b2 selects and outputs an energy change value corresponding to the transition number N which is a random number value generated by the random number generation circuit 114b1.

Functions of the noise table 114b3 will be described later. As the noise table 114b3, for example, a memory such as a RAM, a flash memory, or the like may be used.

The multiplier 114b4 outputs a product obtained by multiplying a value output by the noise table 114b3 by the temperature value T (corresponding to the thermal excitation energy described above).

The comparator 114b5 outputs a comparison result obtained by comparing the multiplication result output by the multiplier 114b4 with the energy change value −ΔE selected by the selector 114b2, as the transition acceptance f.

In the transition control unit 114 illustrated in FIG. 18, the functions described above are basically implemented without change, but a mechanism of accepting a state transition with the acceptance probability expressed by Equation (3-1) will be described in more detail.

A circuit that outputs 1 in a case of the acceptance probability p and outputs 0 in a case of the acceptance probability (1-p) may be implemented by a comparator that has two inputs A and B, outputs 1 when A>B is satisfied, and outputs 0 when A<B is satisfied by inputting the acceptance probability p to the input A and a uniform random number having a value in a section [0, 1) to the input B. Thus, by inputting the value of the acceptance probability p calculated by using Equation (3-1) based on the energy change value and the temperature value T to the input A of the comparator, it is possible to achieve the above function.

For example, when it is assumed that f is a function to be used in Equation (3-1), and that u is a uniform random number having a value in the section [0, 1), the circuit that outputs 1 when f(ΔE/T) is larger than u may achieve the above function.

The same function as that described above may be achieved by any of the following variations.

Even when the same monotonically increasing function is allowed to act on two numbers, the two numbers maintain the same magnitude relationship. Therefore, even when the same monotonically increasing function is allowed to act on the two inputs of the comparator, the same output is obtained. When an inverse function f⁻¹ of f is adopted as this monotonically increasing function, it is seen that a circuit that outputs 1 when −ΔE/T is larger than f⁻¹(u) may be adopted. Since the temperature value T is positive, it is seen that a circuit that outputs 1 when −ΔE is larger than Tf⁻¹(u) is suitable.

The noise table 114b3 illustrated in FIG. 18 is a conversion table for implementing the inverse function f⁻¹(u), and is a table for outputting a value of the next function with respect to the input obtained by discretizing the section [0, 1).

$$\begin{matrix}{f}_{metro}^{- 1} ( u ) = log ( u ) & ( \text{5-1} ) \\ {f}_{Gibbs}^{- 1} ( u ) = log ( \frac{u}{1 - u} ) & ( \text{5-2} )\end{matrix}$$

Although the transition control unit 114 includes a latch that holds a determination result and the like, a state machine that generates the corresponding timing, and the like, these components are not illustrated in FIG. 18 for simple illustration.

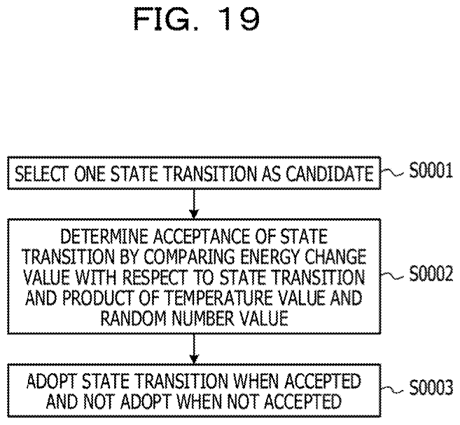

FIG. 19 is a diagram illustrating an example of an operation procedure of the transition control unit 114. The operation procedure illustrated in FIG. 19 includes a step of selecting one state transition as a candidate (S0001), a step of determining whether the state transition is accepted or not by comparing the energy change value with respect to the state transition with a product of a temperature value and a random number value (S0002), and a step (S0003) in which the state transition is adopted when the state transition is accepted, and the state transition is not adopted when the state transition is not accepted.

(Structure Search Method)

The structure search method disclosed herein is, in one embodiment, a structure search method for searching a structure formed with a plurality of molecules having interactions, and the structure search method including structure search processes of

preparing position bits of a number calculated based on the number of constituent units included in a plurality of molecules and the number of molecules included in the plurality of molecules for each constituent unit in each molecule included in the plurality of molecules, and searching the structure formed with the plurality of molecules having the interactions based on a cost function including;
(A-1) a negative interaction that is provided to position bits where respective constituent units in one molecule included in the plurality of molecules are adjacent to each other,
(A-2) an original interaction between the molecules that is provided to position bits when the constituent units in the other molecule included in the plurality of molecules are adjacent to each other,
(B-1) a constraint that each of the constituent units in the one molecule exists in one of the position bits prepared for each of the constituent units in the one molecule, and
(B-2) a constraint that one or no constituent unit in any one of the plurality of molecules exists in the position bits at a same position among the position bits prepared for each of the constituent units in the plurality of molecules.

The structure search method disclosed herein may be performed by, for example, the structure search apparatus disclosed herein. A preferred aspect of the structure search method disclosed herein may be similar to a preferred aspect of the structure search apparatus disclosed herein, for example.

(Structure Search Program)

The structure search program disclosed herein, in one embodiment, is a structure search program for searching a structure formed with a plurality of molecules having interactions, and is executed by a computer, and the structure search program including processes of

preparing position bits of a number calculated based on the number of constituent units included in a plurality of molecules and the number of molecules included in the plurality of molecules for each constituent unit in each molecule included in the plurality of molecules, and searching the structure formed with the plurality of molecules having the interactions based on a cost function including;
(A-1) a negative interaction that is provided to position bits where respective constituent units in one molecule included in the plurality of molecules are adjacent to each other,
(A-2) an original interaction between the molecules that is provided to position bits when the constituent units in the other molecule included in the plurality of molecules are adjacent to each other,
(B-1) a constraint that each of the constituent units in the one molecule exists in one of the position bits prepared for each of the constituent units in the one molecule, and
(B-2) a constraint that one or no constituent unit in any one of the plurality of molecules exists in the position bits at a same position among the position bits prepared for each of the constituent units in the plurality of molecules.

The structure search program disclosed herein may be, for example, a program in which the structure search method as disclosed herein is allowed to be performed by a computer. A preferred aspect of the structure search program disclosed herein may be similar to the preferred aspect of the structure search apparatus disclosed herein.

The structure search program disclosed herein may be created using any of various known program languages according to a configuration of a computer system to be used, and a type, a version, and the like of an operating system.

The structure search program disclosed herein may be recorded on a recording medium such as a built-in hard disk, or an external hard disk, or may be recorded on a recording medium such as a CD-ROM, a DVD-ROM, an MO disk, or a USB memory.

In a case where the structure search program disclosed herein is recorded on the recording medium described above, the structure search program is directly used, or used by installing the structure search program on a hard disk, through a recording medium reading apparatus included in the computer system, as appropriate. The structure search program disclosed herein may be recorded in an external storage area (another computer or the like) accessible from the computer system through an information communication network. In this case, the structure search program disclosed herein and recorded in the external storage area may be directly used or be used by installing the structure search program on the hard disk from the external storage area through the information communication network, as appropriate.

The structure search program disclosed herein may be divided and recorded on a plurality of recording media for each arbitrary process.

(Computer Readable Recording Medium)

The computer readable recording medium disclosed herein is configured to record the structure search program disclosed herein.

The computer readable recording medium disclosed herein is not particularly limited, and may be appropriately selected depending on the purpose, and examples thereof include, for example, a built-in hard disk, an external hard disk, a CD-ROM, a DVD-ROM, an MO disk, a USB memory, and the like.

The computer readable recording medium disclosed herein may be a plurality of recording media in which the structure search program disclosed herein is divided and recorded for each arbitrary process.

### EXAMPLES

Although one example of the technique disclosed herein is described, the technique disclosed herein is not limited to these Examples.

### Example 1-1

As Example 1-1, by using one example of the structure search apparatus disclosed herein, a structure in which eight molecules AB (an example of a polymer) composed of the constituent units A and B were included was searched for a structure whose energy was minimum.

In searching the structure in Example 1-1, the structure search apparatus having the functional configuration illustrated in FIG. 13 was used to search the structure in accordance with the flowchart in FIG. 15.

In Example 1-1, 16 position bits were prepared for each constituent unit in each molecule, and the structure was searched. The position bits were two-dimensionally arranged, and a periodic boundary condition was imposed on the position bits.

As parameters in the above Equation (1), v (the magnitude of the negative interaction) in the first term on the right side of the above Equation (1) was set to −100. As Eₚₐᵢᵣ (the magnitude of the original interaction) of the second term on the right side of the above Equation (1), the interaction V_{AA} between the constituent units A was set to −2, the interaction V_{BB} between the constituent units B was set to −2, and the interaction V_{AB} between the constituent unit A and the constituent unit B was set to −1. p₁ and p₂ in the third and fourth terms on the right side of the above Equation (1) were set to 100.

As the setting of the annealing machine in minimizing the above Equation (2), the calculation was repeated 80001000 times under a condition that the temperature was reduced by 1-0.000201456 times every 1000 times of calculations, and the structure at the time when the temperature became 0.1 was searched.

In Example 1-1, 20 calculations for minimizing the above Equation (2) were performed in parallel, and the structure whose energy became lowest (the value of the cost function became small) among them was made to be the most stable structure.

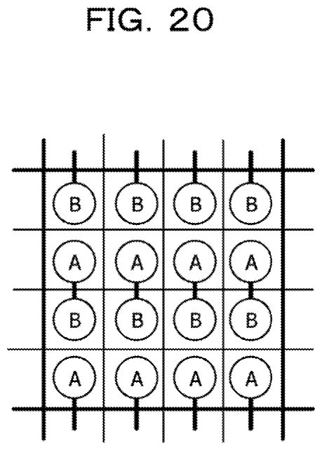

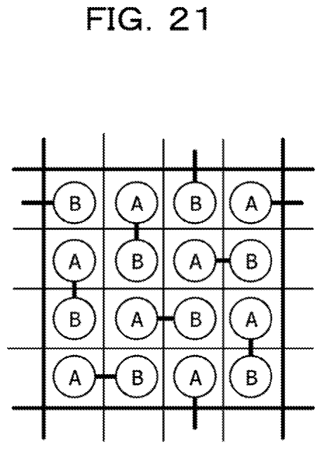

FIG. 20 illustrates a search result of a structure in Example 1-1. As illustrated in FIG. 20, under the conditions of Example 1-1, the structure became a layered structure in which the constituent units A gathered together and the constituent units B gathered together, regularly. The value of the cost function in the structure searched in Example 1-1 was −848.

As illustrated in FIG. 20, since the periodic boundary condition was imposed on the position bits in Example 1-1, the constituent units B arranged in the position bits of the uppermost row in FIG. 20 and the constituent units A arranged in the position bits in the lowermost row in FIG. 20 were combined.

### Example 1-2

In Example 1-2, the structure was searched in the similar manner to Example 1-1, except that the interaction V_{AB} between the constituent unit A and the constituent unit B was set to −3.

FIG. 21 illustrates a search result of the structure in Example 1-2. As illustrated in FIG. 21, the constituent unit A and the constituent unit B were mixed with each other under the conditions of Example 1-2. The value of the cost function in the structure searched in Example 1-2 was −872.

As illustrated in FIG. 21, since the periodic boundary condition was imposed on the position bits in Example 1-2, for example, the constituent unit B arranged in the position bit at the upper left corner in FIG. 21 and the constituent unit A arranged in the position bit at the upper right corner in FIG. 21 were combined.

In this manner, with the technique disclosed herein, in one aspect, a structure formed with a plurality of molecules may be searched in accordance with the properties of the actual molecules, in appropriate consideration of an interaction between the molecules.

### Example 2-1

In Example 2-1, by using an example of the structure search apparatus disclosed herein, searching the structure at a plurality of desired temperatures for the AB alloy in a case where the number of molecules was four, and calculating an energy for each temperature, the transition of the structure due to the change in temperature was analyzed.

In searching the structure in the example 2-1, the structure search apparatus having the functional configuration illustrated in FIG. 13 was used to search the structure in accordance with the flowchart illustrated in FIG. 16. For example, in Example 2-1, the structure with the plurality of molecules were searched at one temperature by repeating decreasing a temperature from a temperature higher than the one temperature to the one temperature a plurality of times to perform averaging, while changing the position bit by using a random number.

In the parameters in the above Equation (1), as Eₚₐᵢᵣ (the magnitude of the original interaction) of the second term on the right side of the Equation (1), the interaction V_{AA} between the constituent units A was set to −2, the interaction V_{BB} between the constituent units B was set to −2, and the interaction V_{AB} between the constituent unit A and the constituent unit B was set to −1. p₁ and p₂ in the third term and the fourth term on the right side of the above Equation (1) were set to 100.

As the setting of the annealing machine in stabilizing the above Equation (2), the calculation was started from the temperature that was 100000 times the desired temperature, and was repeated 60000000 times under the condition that the temperature was multiplied by 0.1 times every 10000000 times of the calculations. The calculation in this setting was repeated 100 to 10000 times (for example, 3072 times) for each desired temperature to calculate the average value of the energies obtained in the respective calculations.

The calculated average value of the energies was plotted for each desired temperature, and the transition of the structure due to the change in temperature was analyzed.

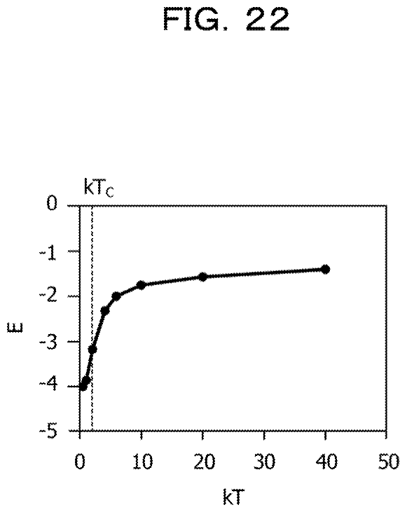

FIG. 22 illustrates an average value of energies for the respective temperatures in Example 2-1. In FIG. 22, the vertical axis represents the average value (E) of the energies, and the horizontal axis represents a temperature (kT).

From FIG. 22, it may be seen that, for example, a phase gradually changes from the regular state to the irregular state at a transition temperature kT_{c} as a boundary. This is considered to be because, in consideration of an entropy of the structure, an irregular state may become stable in a state where the temperature is high.

In this manner, the technique disclosed herein may, in one aspect, change one temperature (desired temperature) to search the structure at a plurality of temperatures to analyze transitions of the structure due to the change in temperature.

### Example 2-2

In Example 2-2, a transition in the structure due to a change in temperature was analyzed in the similar manner to Example 2-1, except that a calculation in which one temperature was held for a certain period of time by a replica exchange method was performed to perform averaging, the structure with the plurality of molecules was searched at the one temperature, and an energy for each temperature was calculated.

Also in Example 2-2, the average value of the energies for the respective temperatures is as illustrated in FIG. 22.

From this, it is understood that, in one example of the technique disclosed herein, the average value of the energies at a desired temperature may be calculated regardless of an averaging method at the one temperature.

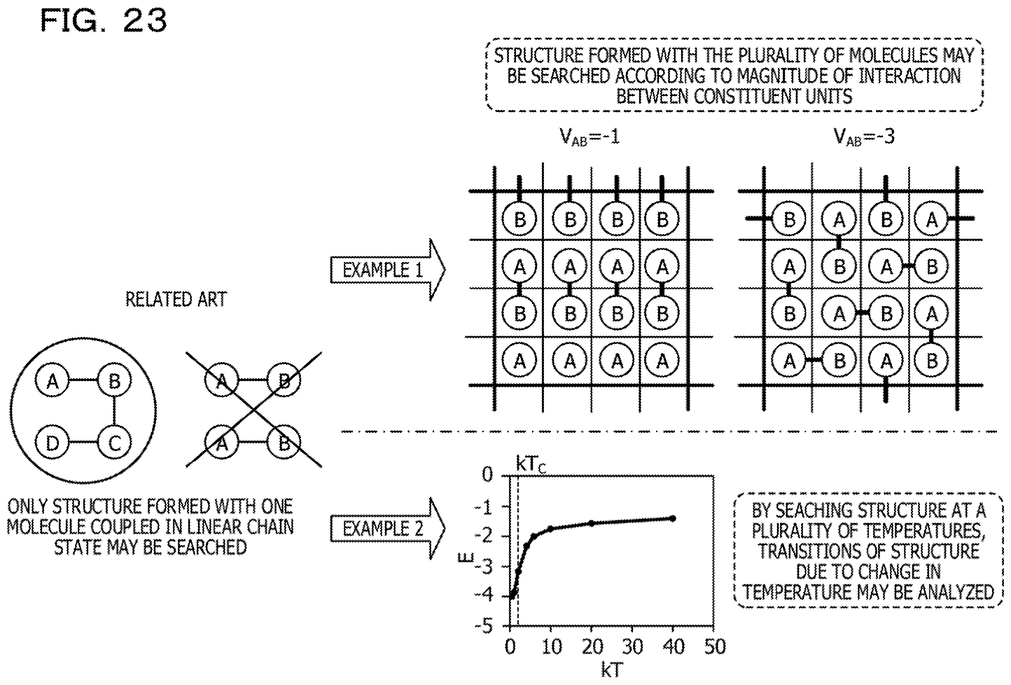

FIG. 23 is a diagram illustrating an example of the structure search of a molecule in each of Examples of the technique disclosed herein and the technique of related art.

As illustrated in FIG. 23, in the related art, only a structure formed with one molecule coupled in a linear chain state may be searched.

On the other hand, the technique disclosed herein, in one aspect, may search the most stable structure with the plurality of molecules whose cost function is minimum according to the magnitude of the interaction between the constituent units. The technique disclosed herein, in one aspect, may analyze the transitions of the structure due to the change in temperature by searching the structure at the plurality of temperatures.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A structure search apparatus for searching a structure formed with a plurality of molecules that have interactions, the structure search apparatus comprising:
a memory, and
a processor coupled to the memory and configured to;
prepare position bits of a number calculated based on a number of constituent units included in the plurality of molecules and a number of molecules included in the plurality of molecules for each constituent unit in each molecule included in the plurality of molecules, and
search the structure formed with the plurality of molecules that have the interactions based on a cost function that includes
(A-1) a negative interaction that is provided to position bits where the respective constituent units in one molecule included in the plurality of molecules are adjacent to each other,
(A-2) an original interaction between the molecules that is provided to the position bits when the constituent units in the other molecule included in the plurality of molecules are adjacent to each other,
(B-1) a constraint that each of the constituent units in the one molecule exists in one of the position bits prepared for each of the constituent units in the one molecule, and
(B-2) a constraint that one or no constituent unit in any one of the plurality of molecules exists in the position bits at a same position among the position bits prepared for each of the constituent units in the plurality of molecules.

2. The structure search apparatus according to claim 1, wherein
when a case where the constituent unit exists in the position bit is set to 1, and a case where the constituent unit does not exist in the position bit is set to 0,
the search step gives a positive cost to the cost function when a sum of the position bits prepared for the respective constituent units in the one molecule is not 1 in the (B-1) constraint.

3. The structure search apparatus according to claim 1, wherein
when a case where the constituent unit exists in the position bit is set to 1, and a case where the constituent unit does not exist is set to 0,
the search step provides a positive cost to the cost function when a sum of the position bits at a same position is not 0 or 1, among the position bits prepared for the respective constituent units in the plurality of molecules in the (B-2) constraint.

4. The structure search apparatus according to claim 1, wherein
the (A-1) negative interaction and the (A-2) original interaction satisfy the following inequality
the (A-1) negative interaction<the (A-2) original interaction.

5. The structure search apparatus according to claim 1, wherein
the (A-1) original interaction is such that
an interaction α between the constituent unit in the one molecule and the constituent unit in the other molecule in a case where the constituent unit in the one molecule and the constituent unit in the other molecule are the same, and
an interaction β between the constituent unit in the one molecule and the constituent unit in the other molecule in a case where the constituent unit in the one molecule and the constituent unit in the other molecule are different from each other, wherein
the interaction α and the interaction β are different from each other.

6. The structure search apparatus according to claim 1, wherein
the structure search unit searches the structure formed with the plurality of molecules based on the cost function expressed by the following Equation (1),
$$E = {\sum }_{{N}_{i} = 0}^{N - 1} {\sum }_{〈 n , {n}^{′} 〉}^{ } {\sum }_{{i}_{p} = 0}^{{N}_{p} - 1} {vx}_{m} {x}_{{m}^{′}} + {\sum }_{〈 {N}_{i} , {N}_{i}^{′} 〉} {\sum }_{n = 0}^{{n}_{{N}_{i}} - 1} {\sum }_{{n}^{′} = 1}^{{n}_{{N}_{i}^{′}} - 1} {E}_{pair} {x}_{m} {x}_{{m}^{′}} + {p}_{1} {\sum }_{m = 0}^{M - 1} {( {\sum }_{i = 0}^{t - 1} {x}_{m} - 1 )}^{2} + {p}_{2} {\sum }_{i = 0}^{t - 1} {( {\sum }_{m = 0}^{M - 1} {x}_{m} - 1 )}^{2} \qquad ( 1 )$$
wherein,
the E is the cost function,
the N is a number of the molecules,
the Nᵢ is a number of the molecule,
the n is a number of the constituent unit in the one molecule,
the Nₚ is a number of the position bits adjacent to each other in the position bits prepared for each of the constituent units in each of the molecules,
the iₚ is a number of each of the position bits adjacent to each other in the position bits provided for each of the constituent units in each of the molecules,
the v is a numerical value that represents a magnitude of the negative interaction,
the xₘ is a binary variable that represents the position bit at an m-th position is 0 or 1,
the n_{NI} is a number of the constituent units in the one molecule,
the Eₚₐᵢᵣ is a numerical value that represents a magnitude of the original interaction,
the p₁ and the p₂ are positive numbers,
the M is a total number of the constituent units,
the t is a number of the position bits prepared for each of the constituent units in each of the molecules, and
the i is a number of each of the position bits provided for each of the constituent units in each of the molecules.

7. The structure search apparatus according to claim 6, wherein
the structure search unit searches the structure formed with the plurality of molecules based on the cost function obtained by converting the Equation (1) to an Ising model expressed by the following Equation (2),
$$E = - {\sum }_{i , j = 0}^{ } {w}_{ij} {x}_{i} {x}_{j} - {\sum }_{i = 0}^{ } {b}_{i} {x}_{i} \qquad ( 2 )$$
wherein,
the wᵢⱼ is a coefficient for weighting between the position bit at an i-th position and the position bit at a j-th position,
the bᵢ is a numerical value that represents a bias for the position bit at the i-th position,
the xᵢ is a binary variable that represents that the position bit at the i-th position is 0 or 1, and
the xⱼ is a binary variable that represents that the position bit at the j-th position is 0 or 1.

8. The structure search apparatus according to claim 1, wherein
the search step searches the structure formed with the plurality of molecules by stabilizing the cost function by an annealing method.

9. The structure search apparatus according to claim 1, wherein
the constituent unit is a group of atoms or an atom.

10. The structure search apparatus according to claim 1, wherein
the position bits are positioned in a lattice shape.

11. The structure search apparatus according to claim 1, wherein
a periodic boundary condition is imposed on the position bits.

12. The structure search apparatus according to claim 11, wherein the periodic boundary condition creates a plurality of shapes, each shape having the position bits arranged within the shape; and
the search applies the cost function to the plurality of shapes.

13. The structure search apparatus according to claim 1, wherein
the processor searches the structure formed with the plurality of molecules at one temperature by repeating decreasing a temperature from a temperature higher than the one temperature to the one temperature a plurality of times to perform averaging, while changing the position bit by using a random number.

14. The structure search apparatus according to claim 1, wherein
the processor searches the structure formed with the plurality of molecules at the one temperature by performing a calculation in which the one temperature is held for a certain period of time by a replica exchange method to perform averaging.

15. The structure search apparatus according to claim 1, wherein
the processor searches the structure formed with the plurality of molecules that have the interactions by minimizing the cost function by an annealing method.

16. The structure search apparatus according to claim 1, wherein the search searches for a stable structure for the plurality of molecules and outputs the stable structure as a candidate for further analysis, the candidate being at least one of a polymer, a protein and a low-molecular compound.

17. The structure search apparatus according to claim 1, wherein
the structure search apparatus is an annealing machine implementing an annealing method to perform a ground state search on an energy function represented by an Ising model, and
the processor outputs a calculation result of the search that represents energies of the structure at a plurality of temperatures.

18. The structure search apparatus according to claim 1, wherein the structure of the plurality of molecules searched is not limited only to single molecules coupled in a linear chain state.

19. A structure search method for searching a structure formed with a plurality of molecules that have interactions, the structure search method comprising:
preparing position bits of a number calculated based on a number of constituent units included in the plurality of molecules and a number of molecules included in the plurality of molecules for each constituent unit in each molecule included in the plurality of molecules, and
searching the structure formed with the plurality of molecules that have the interactions based on a cost function that includes
(A-1) a negative interaction that is provided to position bits where the respective constituent units in one molecule included in the plurality of molecules are adjacent to each other,
(A-2) an original interaction between the molecules that is provided to the position bits when the constituent units in the other molecule included in the plurality of molecules are adjacent to each other,
(B-1) a constraint that each of the constituent units in the one molecule exists in one of the position bits prepared for each of the constituent units in the one molecule, and
(B-2) a constraint that one or no constituent unit in any one of the plurality of molecules exists in the position bits at a same position among the position bits prepared for each of the constituent units in the plurality of molecules.

20. A non-transitory computer-readable recording medium storing a structure search program for searching a structure formed with a plurality of molecules that have interactions, the structure search program is configured to cause a computer to perform a process comprising:
preparing position bits of a number calculated based on a number of constituent units included in the plurality of molecules and a number of molecules included in the plurality of molecules for each constituent unit in each molecule included in the plurality of molecules, and
searching the structure formed with the plurality of molecules that have the interactions based on a cost function that includes
(A-1) a negative interaction that is provided to position bits where the respective constituent units in one molecule included in the plurality of molecules are adjacent to each other,
(A-2) an original interaction between the molecules that is provided to the position bits when the constituent units in the other molecule included in the plurality of molecules are adjacent to each other,
(B-1) a constraint that each of the constituent units in the one molecule exists in one of the position bits prepared for each of the constituent units in the one molecule, and
(B-2) a constraint that one or no constituent unit in any one of the plurality of molecules exists in the position bits at a same position among the position bits prepared for each of the constituent units in the plurality of molecules.
